(19) United States
(12) Patent Application Publication
(10) Pub. No.: US 2008/0070235 A1
(43) Pub. Date: Mar. 20, 2008

(54) Method for Recognizing Acute Generalized Inflammatory Conditions (Sirs), Sepsis, Sepsis-Like Conditions and Systemic Infections

(75) Inventors: Stefan Russwurm, Jena (DE); Konrad Reinhart, Jena (DE); Hans-Peter Saluz, Jena (DE); Eberhard Straube, Jena (DE); Peter F. Zipfel, Jena (DE); Hans-Peter Deigner, Lampertheim (DE)

Correspondence Address: AKERMAN SENTERFITT, P.O. BOX 3188, WEST PALM BEACH, FL, 33402-3188, US

(73) Assignee: SIRS-Lab GMBH, Jena (DE)

(21) Appl. No.: 10/551,874

(22) PCT Filed: Mar. 31, 2004
(86) PCT No.: PCT/EP04/03419

§ 371 Date: May 7, 2007

(30) Foreign Application Priority Data
Apr. 2, 2003 (DE) 103 15 031 5
Aug. 8, 2003 (DE) 103365.7
Sep. 2, 2003 (DE) 103 40 395.7

(51) Int. Cl. C12Q 1/68; C12P 19/34

(52) U.S. Cl. 435/6; 435/7.21; 436/501; 436/94; 435/91.2

(57) ABSTRACT

The present invention relates to a method for in vitro detection of SIRS, sepsis and/or sepsis-like conditions. This method renders the evaluation of the severity and/or the therapeutic progress of sepsis and severe infections, in particular sepsis-like systemic infections possible. Further, the present invention relates to the use of recombinantly or synthetically prepared nucleic acid sequences or peptide sequences derived therefrom as calibrator in sepsis assays and/or for the evaluation of the effect and the toxicity during screening of the active agents and/or the preparation of therapeutics for the prevention and treatment of SIRS, sepsis, sepsis-like systemic inflammatory conditions and sepsis-like systemic infections.

### CROSS REFERENCE TO RELATED APPLICATIONS

This application is a National Stage of International Application PCT/EP04/03419, filed Mar. 31, 2004. International Application PCT/EP04/03419 cites for priority German application numbers 103 15 031.5 (filed Apr. 2, 2003), 103 36 511.7 (filed Aug. 8, 2003), and 103 40 395.7 (filed Sep. 2, 2003). This application incorporates by reference International Application PCT/EP04/03419, German application number 103 15 031.5, German Application Number 103 36 511.7, and German Application Number 103 40 395.7. This application incorporates by reference the Sequence Listing electronically submitted under file name “3535-027SuppSequence.TXT”, with the listed creation date of “May 7, 2007” and being “9,409 KB” in size.

### BACKGROUND OF THE INVENTION

The present invention relates to a method for in vitro detection of acute generalized inflammatory conditions (SIRS), sepsis, sepsis-like conditions, and systemic infections, as well as the use of recombinantly or synthetically prepared nucleic acid sequences or peptide sequences derived therefrom.

Part of the description of the present invention is a sequence listing of 1430 pages, consisting of SEQ ID No: 1 through SEQ ID No: 10,540.

The complete sequence listing is incorporated herein by reference, is part of the description and, thus, part of the disclosure of the present invention.

The present invention particularly refers to labels for gene activity for the diagnosis and for the optimization of the therapy of acute generalized inflammatory conditions (Systemic Inflammatory Response Syndrome (SIRS)). Additionally, the present invention relates to methods for detecting acute generalized inflammatory conditions and/or sepsis, sepsis-like conditions, severe sepsis and systemic infections as well as for a corresponding improvement of therapy of acute generalized inflammatory conditions (SIRS).

Further, for patients suffering from acute generalized inflammatory conditions (SIRS) the present invention relates to new possibilities of diagnosis that are obtained from experimentally proofed findings in connection with the occurrence of changes in gene activity (transcription and subsequent protein expression).

In spite of the fact that there have been improvements of the pathophysiologic understanding and the supportive treatment of patients in intensive care units, SIRS is a disease that occurs very frequently and contributes considerably to mortality in patients in intensive care units [2-5].

The criteria of the American College of Chest Physicians/Society of Critical Care Medicine Consensus Conference (ACCP/SCCM) of 1992 are the ones that became most accepted in the international literature as definition of the term SIRS [4]. According to this definition, SIRS (in this patent described as acute generalized inflammatory conditions) is defined as systemic response of the inflammatory system triggered by a noninfectious stimulus. At least two of the following criteria have to be fulfilled in this context: Fever>38° C. or hypothermia<36° C., leukocytosis>12 G/1 or leukopenia<4 G/1 or shift to the left in the haemogram, heart rate>90/min, tachypnoea>20 breaths/min or PaCO2<4.3 kPa, respectively.

The mortality rate in SIRS amounts to about 20% and increases with the development of more severe organ dysfunctions [6]. The contribution of SIRS to morbidity and lethality is of multidisciplinary interest, as it increasingly puts the success of the most advanced or experimental treatment methods of many medicinal fields (e.g. cardiosurgery, traumatology, transplantation medicine, heamatology/onkology) at a risk, as they all are threatened by an increased risk of the development of an acute generalized inflammatory conditions. Thus, the decrease of morbidity and lethality of many seriously ill patients goes along with the improvement of prevention, treatment and particularly detection and observation of the progress of acute generalized inflammatory conditions.

SIRS is a result of complex and very heterogeneous molecular processes that are characterized by the incorporation of many components and their interactions on every organizational level of the human body: genes, cells, tissues, organs. The complexity of the underlying biological and immunological processes resulted in many kinds of studies comprising a wide range of clinical aspects. One of the results from these studies was that the evaluation of new therapies is rendered more difficult due to the presently used criteria which are quite unspecific and clinical based and which do not sufficiently show the molecular mechanisms [7].

Unfortunately, sepsis and consecutive organ dysfunctions still rank among the principal causes of death in non-cardiologic intensive care units [1-3]. It is supposed that 400,000 patients suffer from sepsis in the USA each year [4]. Lethality is about 40% and increases to 70-80% if a shock develops [5, 6]. The excess lethality independent from the underlying disease of the patient and the underlying infection amounts to 35% [8].

The criteria of the American College of Chest Physicians/Society of Critical Care Medicine Consensus Conference (ACCP/SCCM) of 1992 are the ones that became most accepted in the international literature as definition of the term sepsis [4]. According to these criteria [4] the grades of severity “systemic inflammatory response syndrom” (SIRS), “sepsis”, “severe sepsis” and “septic shock” are clinically defined. According to this definition, SIRS (in this patent described as acute generalized inflammatory conditions) is defined as the systemic response of the inflammatory system triggered by a noninfectious stimulus. At least two of the following criteria have to be fulfilled in this context: Fever>38° C. or hypothermia<36° C., leukocytosis>12G/1 or leukopenia<4G/1 or shift to the left in the haemogram, heart rate>90/min, tachypnoea>20 breaths/min or PaCO2<4.3 kPa, respectively. According to the definition, sepsis are those clinical conditions in which the criteria of SIRS are fulfilled and an infection is detected as cause or it is at least very likely that it is the cause. A severe sepsis is characterized by the additional occurrence of organ dysfunctions. Frequent organ dysfunctions are changes in the state of consciousness, oliguria, lactate acidosis or sepsis-induced hypotension with a systolic blood pressure lower than 90 mmHg, or a pressure decrease of more than 40 mmHg of the initial value, respectively. If such a hypotension cannot be treated by administration of crystalloids and/or colloids and the patient further needs treatment with catecholamines, this is called a septic shock. Such a septic shock is detected in about 20% of all sepsis patients.

Whether and how catecholamines are administered during the treatment of patients suffering from severe sepsis depends on the physician. If the blood pressure decreases, many physicians react by administering large quantities of infusion solutions and, thus, avoid administering catecholamines, however, there are also many physicians who refuse this kind of proceeding and who administer catecholamines much earlier and at a higher dose, if the patient shows the same clinical severity. The consequence is that in everyday practice patients suffering from the same clinical severity can be rated as belonging to the group “severe sepsis” or to the group “septic shock” [4] due to subjective reasons. This is why it became common in international literature to pool patients with the severity grades “severe sepsis” and “septic shock” [4] in one group. This is why the term “severe sepsis” used in this description is used according to the above mentioned consensus conference for patients with sepsis and additional proof of organ dysfunctions and, thus, comprises all patients of the groups “severe sepsis” and “septic shock” according to [4].

The mortality rate in sepsis amounts to about 40% and increases to 70-80%, if a severe sepsis develops [5, 6]. The contribution of sepsis and severe sepsis to morbidity and lethality is of multidisciplinary interest. By comparison, the number of cases rose continuously (by 139% from 73.6 to 176 cases per 100,000 hospital patients from 1970 and 1977, for example) [7]. This increasingly puts the success of the most advanced or experimental treatment methods of many medicinal fields (e.g. visceral surgery, transplantation medicine, heamatology/onkology) at a risk, as they all are threatened by an increased risk of the development of acute generalized inflammatory conditions. Thus, the decrease of morbidity and lethality of many seriously ill patients goes along with a progress in prevention and treatment and especially detection and observation of the progress of the sepsis and severe sepsis. This is why well-known authors have been criticizing for a long time that too much energy and financial resources have been spend on the search for therapeutics for sepsis in the past decade, instead of using them for improving sepsis diagnosis.

Sepsis is a result of complex and highly heterogeneous molecular processes that are characterized by the incorporation of many components and their interactions on every organizational level of the human body: genes, cells, tissues, organs. The complexity of the underlying biological and immunological processes resulted in many kinds of studies comprising a wide range of clinical aspects. One of the results from these studies was that the evaluation of new sepsis therapies is rendered more difficult due to the unspecific clinically based inclusioncriteria, which does not sufficiently show the molecular mechanisms [9].

These facts have created need for innovative diagnostic means that are supposed to improve the capability of the person skilled in the art to diagnose patients suffering from SIRS, sepsis, sepsis-like conditions, severe sepsis and systemic infection at an early stage, to render the severity of a SIRS measurable on a molecular basis and to make it comparable in the clinical progress and to derive information concerning the individual prognosis and the reaction on specific treatments.

The contribution of sepsis with regard to morbidity and lethality is of multidisciplinary interest. Lethality of sepsis changed only marginally within the last decades, whereas, in comparison, the indices increased continuously (e.g. from 1979 to 1987 by 139% from 73.6 to 176 per 100,000 in-patients) [7]. This increasingly puts the success of treatment of the most advanced or experimental therapy methods of various special fields (visceral surgery, transplantation medicine, heamatology/onkology) at a risk due to the fact that they all imply without exception an increase of the risk of sepsis. Thus, the decrease of morbidity and lethality of many seriously ill patients goes along with a progress in prevention and treatment and especially diagnosis of sepsis.

Sepsis is a result highly heterogeneous molecular processes that are characterized by the incorporation of many components and their interactions on every organizational level of the human body: genes, cells, tissues, organs. The complexity of the underlying biological and immunological processes resulted in many kinds of studies comprising a wide range of clinical aspects. One of the results from these studies was that the evaluation of new sepsis therapies is rendered more difficult due to relatively unspecific clinically-based inclusioncriteria which do not sufficiently show the molecular mechanisms [9].

Technological improvements, especially the development of microarray technology, are now rendering it possible for the person skilled in the art to compare 10 000 genes or more and their gene products at the same time. The use of such microarray technologies can now give hints on the conditions of health, regulation mechanisms, biochemical interactions and signalization networks. As the comprehension how an organism reacts to infections is improved this way, this should facilitate the development of enhanced modalities of detection, diagnosis and therapy of systemic disorders.

Microarrays have their origin in “southern blotting” [10], the first approach to immobilize DNA-molecules so that it can be addressed three-dimensionally on a solid matrix. The first microarrays consisted of DNA-fragments, frequently with unknown sequence, and were applied dotwise onto a porous membrane (normally nylon). It was routine to use cDNA, genomic DNA or plasmid libraries, and to mark the hybridized material with a radioactive group [11-13].

Recently, the use of glass as substrate and fluorescence for detection together with the development of new technologies for the synthesis and for the application of nucleic acids in very high densities allowed the miniaturizing of the nucleic acid arrays. At the same time, the experimental throughput and the information content were increased [14-16].

Further, it is known from WO 03/002763 that microarrays basically can be used for the diagnosis of sepsis and sepsis-like conditions.

The first explanation for the applicability of microarray technology was obtained through clinical studies on the field of cancer research. Here, expression profiles proofed to be valuable with regard to identification of activities of individual genes or groups of genes, correlating with certain clinical phenotypes [17]. Many samples of individuals with or without leukemia or diffuse lymphoma of large B-cells were analyzed and gene expression labels (RNA) were found and used for the classification of those kinds of cancer [17, 18]. Golub et al. found out that an individual gene is not enough to make reliable predictions, however, that predictions made on gene expression profiles of 53 genes (selected from more than 6000 genes that were present on the arrays) are highly accurate [17].

Alisadeh et al. [18] examined large B-cell lymphoma (DLBCL). The authors created expression profiles with a “lymph chip”, a microarray bearing 18 000 clones of complementary DNA that was developed to monitor genes that are involved in normal and abnormal development of lymphocytes. By using cluster analysis, they managed to classify DILBCL in two categories that showed great differences with regard to the survival chance of patients. The gene expression profiles of these subtypes corresponded to two important stages of differentiation of B-cells.

To differentiate between symptoms that base on microbial infections and other symptoms of non-infectious etiology, which could indicate sepsis due to their clinical appearance, but are in fact not based on a systemic microbial infection, e.g. of symptoms that base on non-infectious inflammation of individual organs, the determination of gene expression profiles via differential diagnostics proofed to be particularly advantageous [19-22]. The use of body fluids for the measurement of gene expression profiles for the diagnosis of SIRS has not yet been described.

The point of origin of the invention disclosed in the present patent application is the realization that RNA levels different from normal values respectively peptide levels or peptide segment levels derivable from the RNA levels, that can be detected in a serum or plasma of a patient whose risk is high that he will be suffering from SIRS, or who suffers from symptoms that are typical for SIRS, can be detected before SIRS, sepsis, sepsis-like conditions, severe sepsis and systemic Infections are detected in biological samples.

Thus, it is an object of the present invention to provide a method for the detection, evaluation of the degree of severity, and/or the progress of the therapy, of SIRS and/or sepsis and/or severe sepsis and/or systemic infections.

The method of the invention is characterized in that the activity of one or more leading genes can be determined in a sample of a biological liquid of an individual. Additionally, SIRS and/or the success of a therapeutic treatment can be deduced from the presence and/or, if present, the amount of the determined gene product.

One embodiment of the present invention is characterized in that the method for in vitro detection of SIRS comprises the following steps:
a) Isolation of sample RNA from a sample of a mammal;
b) Labelling of the sample RNA and/or at least one DNA being a gene or gene fragment specific for SIRS, with a detectable label.
c) Contacting the sample RNA with the DNA under hybridization conditions;
d) Contacting control RNA representing a control for non-pathologic conditions, with at least one DNA, under hybridization conditions, whereby the DNA is a gene or gene fragment specific for SIRS;
e) Quantitative detection of the label signals of the hybridized sample RNA and control RNA;
f) Comparing the quantitative data of the label signals in order to determine whether the genes or gene fragments specific for SIRS are more expressed in the sample than in the control, or less.

One alternative embodiment of the present invention is characterized in that the method for in vitro detection of sepsis and/or sepsis-like conditions comprises the following steps:
g) Isolation of sample RNA from a sample of a mammal;
h) Labelling of the sample RNA and/or at least one DNA being a specific gene or gene fragment for sepsis and/or sepsis-like conditions, with a detectable label.
i) Contacting the sample RNA with the DNA under hybridization conditions;
j) Contacting sample RNA representing a control for non-pathologic conditions, with at least one DNA, under hybridization conditions, whereby the DNA is a gene or gene fragment specific for sepsis and/or sepsis-like conditions;
k) Quantitative detection of the label signals of the hybridized sample RNA and control RNA;
l) Comparing the quantitative data of the marking signals in order to determine whether the genes or gene fragments specific for sepsis and/or sepsis-like conditions are more expressed in the sample than in the control, or less.

One embodiment of the present invention is characterized in that the method for in vitro detection of severe sepsis comprises the following steps:
m) Isolation of sample RNA from a sample of a mammal;
n) Labelling of the sample RNA and/or at least one DNA being a specific gene or gene fragment for severe sepsis, with a detectable label.
o) Contacting the sample RNA with the DNA under hybridization conditions;
p) Contacting sample RNA representing a control for non-pathologic conditions, with at least one DNA, under hybridization conditions, whereby the DNA is a gene or gene fragment specific for severe sepsis;
q) Quantitative detection of the label signals of the hybridized sample RNA and control RNA;
r) Comparing the quantitative data of the label signals in order to determine whether the genes or gene fragments specific for severe sepsis are more expressed in the sample than in the control, or less.

A further embodiment of the present invention is characterized in that the control RNA is hybridized with the DNA before the measurement of the sample RNA and the label signals of the control RNA/DNA complex is gathered and, if necessary, recorded in form of a calibration curve or table.

Another embodiment of the present invention is characterized in that mRNA is used as sample RNA.

Another embodiment of the present invention is characterized in that the DNA is arranged, particularly immobilized, on predetermined areas on a carrier in form of a microarray.

Another embodiment of the invention is characterized in that the method is used for early detection by means of differential diagnostics, for control of the therapeutic progress, for risk evaluation for patients as well as for post mortem diagnosis of SIRS and/or sepsis and/or severe sepsis and/or systemic infections and/or septic conditions and/or infections.

Another embodiment of the present invention is characterized in that the sample is selected from: body fluids, in particular blood, liquor, urine, ascitic fluid, seminal fluid, saliva, puncture fluid, cell content, or a mixture thereof.

Another embodiment of the present invention is characterized in that cell samples are subjected a lytic treatment, if necessary, in order to free their cell contents.

Another embodiment of the present invention is characterized in that the mammal is a human.

Another embodiment of the invention is characterized in that the gene or gene segment specific for SIRS is selected from the group consisting of SEQ. ID No. 6373 to SEQ. ID No. 10540, as well as from gene fragments thereof having at least 5-2000, preferably 20-200, more preferably 20-80 nucleotides.

Another embodiment of the invention is characterized in that the gene or gene segment specific for sepsis and/or sepsis-like conditions is selected from the group consisting of SEQ. ID No. 1 to SEQ. ID No. 6242, as well as gene fragments thereof with 5-2000 or more, preferably 20-200, more preferably 20-80 nucleotides.

Another embodiment of the invention is characterized in that the gene or gene segment specific for severe sepsis is selected from the group consisting of SEQ. ID No. 6243 to SEQ. ID No. 6372, as well as gene fragments thereof with 5-2000 or more, preferably 20-200, more preferably 20-80 nucleotides.

Another embodiment of the present invention is characterized in that the immobilized probes are labelled. As probes for this embodiment serve self-complementary oligonucleotides, so called molecular beacons. They bear a fluorophore/quencher pair at their ends, so that they are present in a folded hairpin structure and only deliver a fluorescence signal with corresponding sample sequence. The hairpin structure of the molecular beacons is stable until the sample hybridizes at the specific catcher sequence, this leading to a change in conformation and, thus, to the release of reporter fluorescence.

Another embodiment of the present invention is characterized in that at least 2 to 100 different cDNAs are used.

Another embodiment of the present invention is characterized in that at least 200 different cDNAs are used.

Another embodiment of the present invention is characterized in that at least 200 to 500 different cDNAs are used.

Another embodiment of the present invention is characterized in that at least 500 to 1000 different cDNAs are used.

Another embodiment of the present invention is characterized in that at least 1000 to 2000 different cDNAs are used.

Another embodiment of the present invention is characterized in that the cDNA of the genes listed in claim 10 is replaced by synthetic analoga as well as peptidonucleic acids.

Another embodiment of the present invention is characterized in that the synthetic analoga of the genes comprise 5-100, in particular about 70 base pairs.

Another embodiment of the present invention is characterized in that a radioactive label is used as detectable label, in particular ³²P, ¹⁴C, ¹²⁵I, ¹⁵⁵Eu, ³³P or ³H.

Another embodiment of the present invention is characterized in that a non-radioactive label is used as detectable label, in particular a color- or fluorescence label, an enzyme label or immune label, and/or quantum dots or an electrically measurable signal, in particular the change in potential, and/or conductivity and/or capacity during hybridizations.

Another embodiment of the present invention is characterized in that the sample RNA and control RNA bear the same label.

Another embodiment of the present invention is characterized in that the sample RNA and control RNA bear different labels.

Another embodiment of the present invention is characterized in that the cDNA probes are immobilized on glass or plastics.

Another embodiment of the present invention is characterized in that the individual cDNA molecules are immobilized onto the carrier material by means of a covalent binding.

Another embodiment of the present invention is characterized in that the individual cDNA molecules are immobilized onto the carrier material by means of adsorption, in particular by means of electrostatic and/or dipole-dipole and/or hydrophobic interactions and/or hydrogen bridges.

Another embodiment of the method according to the present invention for in vitro detection of SIRS is characterized in that it comprises the following steps:
a) Isolation of sample peptides from a sample of a mammal;
b) Labelling of the sample peptides with a detectable label;
c) Contacting the labelled sample peptides with at least one antibody or its binding fragment, whereby the antibody binds a peptide or peptide fragment specific for SIRS;
d) Contacting the labelled control peptides originating from healthy subjects, with at least one antibody or its binding fragment immobilized in form of a microarray on a carrier, whereby the antibody binds a peptide or peptide fragment specific for SIRS;
e) Quantitative detection of the label signals of the sample peptides and the control peptides;
f) Comparing the quantitative data of the label signals in order to determine whether the genes or gene fragments specific for SIRS are more expressed in the sample than in the control, or less.

Another alternative embodiment of the method according to the present invention for in vitro detection of sepsis and/or sepsis-like conditions is characterized in comprising the following steps:
g) Isolation of sample peptides from a sample of a mammal;
h) Labelling of the sample peptides with a detectable label;
i) Contacting the labelled sample peptides with at least one antibody or its binding fragment, whereby the antibody binds a peptide or peptide fragment specific for sepsis and/or sepsis-like conditions;
j) Contacting the labelled control peptides originating from healthy subjects, with at least one antibody or its binding fragment immobilized on a carrier in form of a microarray, whereby the antibody binds a peptide or peptide fragment specific for sepsis and/or sepsis-like conditions;
k) Quantitative detection of the label signals of the sample peptides and the control peptides;
l) Comparing the quantitative data of the label signals in order to determine whether the genes or gene fragments specific for sepsis and/or sepsis-like conditions are more expressed in the sample than in the control, or less.

Another embodiment of the method according to the present invention for in vitro detection of severe sepsis is characterized in comprising the following steps:
m) Isolation of sample peptides from a sample of a mammal;
n) Labelling of the sample peptides with a detectable label;
o) Contacting the labelled sample peptides with at least one antibody or its binding fragment, whereby the antibody binds a peptide or peptide fragment specific for severe sepsis;
p) Contacting the labelled control peptides stemming from healthy subjects, with at least one antibody or its binding fragment immobilized on a carrier in form of a microarray, whereby the antibody binds a peptide or peptide fragment specific for severe sepsis;
q) Quantitative detection of the label signals of the sample peptides and the control peptides;
r) Comparing the quantitative data of the label signals in order to determine whether the genes or gene fragments specific for severe sepsis are more expressed in the sample than in the control, or less.

Another embodiment of the present invention is characterized in that the antibody is immobilized on a carrier in form of a microarray.

Another embodiment of the present invention is characterized in providing an immunoassay.

Another embodiment of the invention is characterized in that the method is used for early detection by means of differential diagnostics, for control of the therapeutic progress, for risk evaluation for patients as well as for post mortem diagnosis of SIRS and/or sepsis and/or severe sepsis and/or systemic infections.

Another embodiment of the present invention is characterized in that the sample is selected from: body fluids, in particular blood, liquor, urine, ascitic fluid, seminal fluid, saliva, puncture fluid, cell content, or a mixture thereof.

Another embodiment of the present invention is characterized in that tissue- and cell samples are subjected to a lytic treatment, if necessary, in order to free the content of the cells.

Another embodiment of the present invention is characterized in that the mammal is a human.

Another embodiment of the invention is characterized in that the peptide specific for SIRS is an expression product of a gene or gene fragment selected from the group consisting of SEQ. ID No. 6373 to SEQ. ID No. 10540, as well as gene fragments thereof with 5-2000 or more, preferably 20-200, more preferably 20-80 nucleotides.

Another embodiment of the invention is characterized in that the peptide specific for sepsis and/or sepsis-like conditions is an expression product of a gene or gene fragment selected from the group consisting of SEQ. ID No. 1 to SEQ. ID No. 6242, as well as gene fragments thereof with 5-2000 nucleotides or more, preferably 20-200, more preferable 20-80 nucleotides.

Another embodiment of the invention is characterized in that the peptide specific for severe sepsis is an expression product of a gene or gene fragment selected from the group consisting of SEQ. ID No. 6243 to SEQ. ID No. 6372, as well as gene fragments thereof with 5-2000 or more, preferably 20-200, more preferably 20-80 nucleotides.

Another embodiment of the present invention is characterized in that at least 2 to 100 different peptides are used.

Another embodiment of the present invention is characterized in that at least 200 different peptides are used.

Another embodiment of the present invention is characterized in that at least 200 to 500 different peptides are used.

Another embodiment of the present invention is characterized in that at least 500 to 1000 different peptides are used.

Another embodiment of the present invention is characterized in that at least 1000 to 2000 different peptides are used.

Another embodiment of the present invention is characterized in that a radioactive label is used as detectable label, in particular ³²P, ¹⁴C, ¹²⁵I, ¹⁵⁵Eu, ³³P or ³H.

Another embodiment of the present invention is characterized in that a non-radioactive label is used as detectable label, in particular a color- or fluorescence label, an enzyme label or immune label, and/or quantum dots or an electrically measurable signal, in particular the change in potential, and/or conductivity and/or capacity during hybridizations.

Another embodiment of the present invention is characterized in that the sample peptides and control peptides bear the same label.

Another embodiment of the present invention is characterized in that the sample peptides and control peptides bear different labels.

Another embodiment of the present invention is characterized in that the peptide probes are immobilized on glass or plastics.

Another embodiment of the present invention is characterized in that the individual peptide molecules are immobilized onto the carrier material by means of a covalent binding.

Another embodiment of the present invention is characterized in that the individual peptide molecules are immobilized on the carrier material by means of adsorption, in particular by means of electrostatic and/or dipole-dipole and/or hydrophobic interactions and/or hydrogen bridges.

Another embodiment of the present invention is characterized in that the individual peptide molecules are detected by means of monoclonal antibodies or their binding fragments.

Another embodiment of the present invention is characterized in that the determination of individual peptides by means of immunoassay or precipitation assay is carried out using monoclonal antibodies.

Another embodiment of the present invention is the use of recombinantly or synthetically produced nucleic acid sequences, partial sequences or protein-/peptide-sequences derived thereof, specific for SIRS, individually or as partial quantities as calibrator in SIRS-assays and/or to evaluate the effects and toxicity when screening for active agents and/or for the preparation of therapeutics as well as of substances and compounds that are designed to act as therapeutics, for prevention and treatment of SIRS.

Another embodiment of the present invention is the use of recombinantly or synthetically produced nucleic acid sequences, partial sequences or protein-/peptide-sequences derived thereof, specific for sepsis and/or sepsis-like conditions, individually or as partial quantities as calibrator in sepsis-assays and/or to evaluate the effects and toxicity when screening for active agents and/or for the preparation of therapeutics as well as of substances and compounds that are designed to act as therapeutics, for prevention and treatment of sepsis, sepsis-like systemic inflammatory conditions and sepsis-like systemic infections.

Another embodiment of the present invention is the use of recombinantly or synthetically produced nucleic acid sequences, partial sequences or protein-/peptide-sequences derived thereof, specific for severe sepsis, individually or as partial quantities as calibrator in sepsis-assays and/or to evaluate the effects and toxicity when screening for active agents and/or for the preparation of therapeutics as well as of substances and compounds that are designed to act as therapeutics, for prevention and treatment of severe sepsis.

It is obvious to the person skilled in the art that the individual features of the present invention shown in the claims can be combined with each other in any desired way.

The term leading genes as used in the present invention means all derived DNA-sequences, partial sequences and synthetic analoga (for example peptido-nucleic acids, PNA). In the present invention, it further means all proteins, peptides or partial sequences, respectively, or synthetic peptide mimetics decoded by leading genes are meant. The description of the invention referring to the determination of the gene expression is not a restriction but only an exemplary application of the present invention.

The description of the invention referring to blood is only an exemplary embodiment of the present invention. The term biological liquids as used in the present invention means all human body fluids.

One application of the method according to the invention is the measurement of differential gene expression with SIRS, sepsis, sepsis-like conditions, severe sepsis and systemic infections. For this measurement, the RNA is isolated from the whole blood of corresponding patients and a control sample of a healthy subject or of a subject that is not suffering from one of the above-mentioned disorders. Subsequently, the RNA is labelled, for example radioactively with ³²P or with dye molecules (fluorescence). All molecules and/or detection signals known in the state of the art for labelling molecules may be used as labelling molecules. The person skilled in the art is also aware of the corresponding molecules and/or methods.

The RNA thus labelled is subsequently hybridized with cDNA-molecules that are immobilized on a microarray. The cDNA-molecules immobilized on the microarray are a specific selection of genes according to claim 12 of the present invention for the measurement of SIRS, according to claim 13 for sepsis and sepsis-like conditions, according to claim 14 for severe sepsis and systemic infections.

The intensity signals of the hybridized molecules are measured afterwards by means of suitable instruments (phosphorimager, microarray scanner) and analyzed by means of additional computer-based analysis. The expression ratios of the sample of the patient and the control are determined with the signal intensities measured. The expression ratios of the under- and/or overregulated genes indicate, as in the experiments described below, whether SIRS, sepsis, sepsis-like conditions, severe sepsis and systemic infections are present or not.

Another use of the method according to the invention is the measurement of the differential gene expression to determine how probable it is that the patient will respond to the planned therapy, and/or for determination of the reaction to a specialized therapy and/or the settlement of the end of the therapy in terms of a “drug monitoring” with patients suffering from SIRS, sepsis, sepsis-like conditions, severe sepsis and systemic infections. For this purpose, the RNA (sample RNA) is isolated from the blood samples of the patient, that have been taken in time intervals. The different RNA samples are labelled together with the control sample and hybridized with the selected genes that are immobilized on a microarray. Thus, the corresponding expression ratios show the probability that patients respond to the planned therapy, and/or whether the started therapy is effective, and/or how long the patients' treatment has to go on, and/or whether the maximum effect of the therapy has already been achieved with the dose and duration applied.

Another use of the method according to the invention is the measurement of the binding grade of proteins, for example monoclonal antibodies, by means of the use of immunoassays, protein- and peptide arrays or precipitation assays. Durch die Bestimmung der Konzentration der von den Sequenzen der in Anwendungsbeispiel 1 aufgeführten Nukleinsäuren entsprechenden Proteine or Peptide kann auf ein erhöhtes Risiko zur Entwicklung einer SIRS geschlossen werden. Additionally, this procedure allows the differential diagnostic determination in patients suffering from SIRS, sepsis, sepsis-like conditions, severe sepsis and systemic infections.

Additionally, this indicates a higher risk of development of sepsis, sepsis-like conditions, severe sepsis and systemic infections.

Further advantages and features of the present invention will become apparent from the description of the embodiments as well as from the drawing.

### BRIEF DESCRIPTION OF THE DRAWINGS

FIG. 1 is a 2-dimensional gel comprising a precipitated serum protein of a patient suffering from sepsis that is applied to it.

FIG. 2 is a 2-dimensional gel comprising a precipitated serum protein of a control that is applied to it.

### DETAILED DESCRIPTION OF THE PREFERRED EMBODIMENTS

### Embodiment 1

### SIRS

Studies of differential gene expression with patients suffering from SIRS.

Whole blood samples of patients who were under the care of a surgical intensive care unit were examined for the measurement of the differential gene expression in connection with SIRS.

Control samples were whole blood samples of the patients that were drawn immediately before the operation. No one of these patients showed an infection and/or clinical signs of SIRS (defined according to the SIRS-criteria [4]) at this point of time or before the stationary treatment.

Additionally, whole blood samples of the same patients who had been subjected to a surgery, were drawn four hours after the operation (patient samples). Each of these patients developed SIRS after the surgery. A range of characteristics of the patients suffering from SIRS are shown in table 1. In Table 1, data with regard to age, gender, diagnosis as well as duration of the extracorporeal treatment are given.

TABLE 1
Data of the group of patients

| | | | | Duration of |
|---|---|---|---|---|
| | | | | extracorporeal |
| Patient | Gender | Age | Diagnosis | treatment [min] |
| 1 | male | 57 | coronary heart disease | 82 |
| 2 | male | 70 | coronary heart disease | 83 |
| 3 | female | 67 | coronary heart disease | 72 |
| 4 | male | 70 | coronary heart disease | 55 |

After the whole blood had been drawn, the total RNA was isolated using the PAXGene Blood RNA Kit according to the producer's (Quiagen) instructions. Subsequently, the cDNA was synthesized from the total RNA by means of reverse transcriptions with Superscript II RT (Invitrogen) according to the producer's instructions, labelled with aminoallyl-dUTP and succinimidylester of the fluorescent dyes Cy3 and Cy5 (Amersham), and hydrolyzed.

The microarrays (Lab-Arraytor human 500-1 cDNA) of the company SIRS-Lab GmbH were used for the hybridization. These micorarrays are loaded with 340 humane cDNA-molecules. The 340 humane cDNA-molecules are 3-fold immobilized in three subarrays on each microarray.

The prepared and labelled samples were hybridized with the microarrays according to the producer's instructions and subsequently washed. The fluorescence signals of the hybridized molecules were measured by means of a scanner (AXON 4000B).

Analysis

One test was analyzed by means of scanned pictures of the microarrays after hybridization. The mean intensity value of the detected spots was defined as the measured expression value of the corresponding gene. Spots were automatically identified and their homogeneity was checked. The analysis was controlled manually. In addition to the desired information, namely the amount of nucleic acids bound, contain the detected signals also background signals which are caused by unspecific bindings to the surface of the membrane. The definition of the signals of the background rendered the optimum differentiation between spots and the surface of the chip possible, which also showed color effects. For the analysis of the microarrays blank spots were chosen as background. The mean expression value of the chosen blank spots within one block (of 14 times 14 spots) was subtracted from the expression values of the gene spots (in the corresponding block).

Point signals not caused by binding of nucleic acids but by dust particles or other disturbances on the filter, could be told from real spots because of their irregular shape and were excluded from further analysis.

In order to render the values between the 3 subarrays and between different microarrays comparable, it was necessary to normalize the data afterwards. Due to the high number of spots on the microarray, the mean value of all expression values was set as normalization reference. The mean expression per gene was calculated by choosing the two (from three) repetitions that were closest to each other.

The expression ratios of the samples of the control and the patients were calculated from the signal intensities using the software AIDA Array Evaluation. The criteria for the grading of the examined genes was the level of the expression ratio. The interesting genes were those which were most overexpressed or underexpressed, respectively, compared with the control samples.

Table 2 shows that 57 genes of the patient sample were found, which were significantly overexpressed, if compared with the control sample. Table 3 shows that 16 genes of the patient sample were found, which were significantly underexpressed, if compared with the control sample. Those results show that the genes listed in table 2 and table 3 correlate with the occurrence of SIRS. Thus, the gene activities of the genes mentioned are labels for a diagnosis of SIRS.

TABLE 2
Significantly increased transcription activities and
their relative ratio to the control sample in SIRS

| GenBank | | | | | | SEQUENCE- |
|---|---|---|---|---|---|---|
| Accession-No. | Hugo-Name | Patient 1 | Patient 2 | Patient 3 | Patient 4 | ID |
| XM_051958 | ALOX5 | 2.43 | 1.49 | 1.81 | 1.40 | 6408 |
| XM_015396 | ALOX5AP | 3.71 | 7.39 | 3.89 | 2.68 | 6409 |
| XM_008738 | BCL2 | 1.16 | 6.76 | 1.55 | 1.04 | 6410 |
| BC016281 | BCL2A1 | 13.71 | 10.29 | 1.41 | 4.36 | 6468 |
| NM_021073 | BMP5 | 2.02 | 1.83 | 1.78 | 1.51 | 6411 |
| XM_002101 | BMP8 | 2.32 | 10.85 | 1.31 | 0.87 | 6412 |
| XM_045933 | CAMKK2 | 2.20 | 1.26 | 1.95 | 1.13 | 6413 |
| XM_055386 | CASP1 | 1.40 | 1.76 | 1.89 | 1.45 | 6414 |
| NM_004347 | CASP5 | 1.92 | 2.77 | 0.67 | 1.89 | 6415 |
| NM_004166 | CCL14 | 1.24 | 1.58 | 2.46 | 0.77 | 6463 |
| XM_012649 | SCYA7 | 1.24 | 9.78 | 0.85 | 1.82 | 6465 |
| NM_001760 | CCND3 | 1.23 | 2.68 | 1.56 | 1.12 | 6416 |
| NM_000591 | CD14 | 3.45 | 4.43 | 1.76 | 2.05 | 6417 |
| XM_038773 | CD164 | 0.84 | 1.91 | 3.26 | 3.15 | 6418 |
| XM_048792 | CD1A | 3.24 | 3.10 | 1.00 | 1.11 | 6419 |
| NM_001779 | CD58 | 2.14 | 2.11 | 1.54 | 2.91 | 6420 |
| XM_002948 | CD80 | 1.69 | 1.16 | 2.25 | 0.69 | 6423 |
| XM_027978 | CFLAR | 2.33 | 4.97 | 1.44 | 1.39 | 6424 |
| NM_000760 | CSF3R | 1.55 | 1.47 | 1.81 | 1.02 | 6425 |
| XM_012717 | CSNK1D | 1.95 | 3.15 | 1.24 | 1.32 | 6426 |
| XM_048068 | SCYD1 | 3.70 | 12.12 | 0.86 | 3.88 | 6466 |
| XM_051229 | CXCR4 | 2.33 | 2.10 | 2.15 | 1.60 | 6427 |
| XM_039625 | DUSP10 | 2.49 | 3.77 | 0.90 | 1.10 | 6429 |
| XM_010177 | DUSP9 | 2.17 | 5.27 | 1.12 | 1.63 | 6430 |
| XM_055699 | ENTPD1 | 1.91 | 3.18 | 0.71 | 0.86 | 6431 |
| XM_007189 | FOXO1A | 1.61 | 3.10 | 1.09 | 1.67 | 6432 |
| XM_012039 | FUT4 | 1.55 | 5.07 | 1.88 | 0.93 | 6433 |
| XM_040683 | HPRT1 | 5.15 | 66.19 | 1.44 | 2.28 | 6434 |
| NM_017526 | OBRGRP | 1.93 | 1.10 | 1.53 | 1.40 | 6435 |
| XM_049516 | ICAM1 | 1.27 | 1.88 | 2.05 | 1.30 | 6436 |
| XM_049531 | ICAM3 | 2.31 | 2.32 | 1.61 | 1.45 | 6437 |
| XM_041744 | IER3 | 4.17 | 7.25 | 1.98 | 2.08 | 6438 |
| XM_048562 | IFNAR1 | 2.16 | 4.87 | 1.09 | 2.36 | 6439 |
| XM_006447 | IL10RA | 1.02 | 1.51 | 1.96 | 0.67 | 6440 |
| M90391 | IL-16 | 1.77 | 1.50 | 1.16 | 1.09 | 6441 |
| XM_002765 | IL1R2 | 2.84 | 12.75 | 1.03 | 2.75 | 6442 |
| NM_000418 | IL4R | 3.34 | 6.44 | 2.05 | 2.79 | 6443 |
| XM_057491 | IL6 | 1.72 | 1.48 | 1.53 | 1.37 | 6444 |
| NM_002184 | IL6ST | 2.50 | 9.25 | 1.07 | 1.87 | 6445 |
| NM_000634 | IL8RA | 2.27 | 3.73 | 1.45 | 1.68 | 6446 |
| NM_006084 | ISGF3G | 1.72 | 1.08 | 2.54 | 1.12 | 6447 |
| XM_045985 | ITGA2B | 3.69 | 2.00 | 0.83 | 3.79 | 6448 |
| XM_008432 | ITGA3 | 2.11 | 7.62 | 1.08 | 1.06 | 6449 |
| XM_028642 | ITGA5 | 2.49 | 4.48 | 1.39 | 3.54 | 6450 |
| XM_036107 | ITGB2 | 1.72 | 1.13 | 2.08 | 1.13 | 6451 |
| XM_009064 | JUNB | 2.21 | 1.84 | 3.59 | 2.05 | 6452 |
| XM_036154 | LAMP2 | 1.79 | 1.68 | 1.62 | 1.41 | 6453 |
| XM_042066 | MAP3K1 | 2.06 | 7.67 | 2.91 | 8.93 | 6454 |
| NM_001315 | MAPK14 | 2.50 | 12.01 | 0.90 | 4.20 | 6455 |
| NM_003684 | MKNK1 | 2.58 | 17.17 | 1.74 | 1.83 | 6456 |
| U68162 | MPL | 2.58 | 1.10 | 1.39 | 6.99 | 6457 |
| NM_004555 | NFATC3 | 1.40 | 1.70 | 2.80 | 0.75 | 6458 |
| XM_006931 | OLR1 | 1.53 | 5.01 | 1.10 | 3.16 | 6459 |
| XM_039764 | PDCD5 | 1.11 | 3.09 | 1.21 | 1.95 | 6460 |
| XM_029791 | PIK3C2G | 0.93 | 1.62 | 0.96 | 1.52 | 6461 |
| NM_006219 | PIK3CB | 1.52 | 0.99 | 0.94 | 1.66 | 6467 |
| XM_043864 | PIK3R1 | 1.81 | 4.07 | 1.48 | 1.26 | 6462 |

TABLE 3
Significantly reduced transcription activities and
their relative ratio to the control sample in SIRS

| GenBank | | | | | | SEQUENCE- |
|---|---|---|---|---|---|---|
| Accession-No. | HUGO Name | Patient 1: | Patient 2: | Patient 3: | Patient 4: | ID |
| BC001374 | CD151 | 0.00 | 0.00 | 0.39 | 0.71 | 6375 |
| XM_006454 | CD3G | 0.63 | 0.40 | 0.75 | 1.01 | 6378 |
| XM_043767 | CD3Z | 0.43 | 0.00 | 0.82 | 0.77 | 6379 |
| XM_056798 | CD81 | 0.50 | 1.12 | 0.32 | 0.00 | 6380 |
| M26315 | CD8A | 1.45 | 0.00 | 0.30 | 1.31 | 6381 |
| NM_004931 | CD8B1 | 0.40 | 0.90 | 0.50 | 1.19 | 6382 |
| NM_001511 | CXCL1 | 0.09 | 0.00 | 0.50 | 1.34 | 6385 |
| XM_057158 | ADCY6 | 1.17 | 0.00 | 0.42 | 1.34 | 6383 |
| XM_044428 | ICAM2 | 0.00 | 1.16 | 0.50 | 1.10 | 6386 |
| NM_000880 | IL7 | 0.00 | 1.06 | 0.74 | 0.10 | 6388 |
| L34657 | PECAM-1 | 0.68 | 0.39 | 1.13 | 0.64 | 6396 |
| XM_044882 | PTGS1 | 0.00 | 1.34 | 0.52 | 0.76 | 6397 |
| XM_035842 | SCYA5 | 0.60 | 0.50 | 0.80 | 0.99 | 6401 |
| NM_021805 | SIGIRR | 0.00 | 0.40 | 0.45 | 0.66 | 6402 |
| XM_057372 | TNFRSF5 | 0.00 | 0.49 | 0.59 | 1.03 | 6406 |
| NM_003809 | TNFSF12 | 1.34 | 0.99 | 0.53 | 0.60 | 6407 |

These characteristic changes can be used for the method according to the present invention.

In the appended sequence listing, which is part of the present invention, the gene bank accession numbers indicated in tables 2 and 3 (access via internet via http://www.ncbi.nlm.nih.gov/) of the individual sequences are each allocated to one sequence ID.

### Embodiment 2

### SIRS

Study of the gene expression of three patients suffering from SIRS, and one control.

The gene expression of three patients suffering from SIRS and one control were measured. All patients developed SIRS as described in the criteria according to [4]. The control sample was taken from one patient who was subjected to surgical treatment, but who did not show any SIRS during this stationery treatment. The date of the patients suffering from SIRS and the control are summarized in table 4.

TABLE 4
Characteristics of the samples of patients and controls

| | | | | Apache | |
|---|---|---|---|---|---|
| | | | | Score | SAPS II |
| Patient | Gender | Age | Diagnosis | [point] | [point] |
| 1 | male | 50 | coronary heart | 18 | 36 |
| | | | disease | | |
| 2 | male | 70 | caecuM_perforation | 19 | 64 |
| 3 | male | 67 | aortic valve | 9 | 21 |
| | | | insuffiency | | |
| 1 | male | 70 | fracture of the | 1 | 12 |
| | | | skull cap | | |

After the whole blood had been drawn, the total RNA was isolated using the RNAeasy-Kit according to the producer's (Quiagen) instructions. Subsequently, the cDNA was synthesized from the total RNA by means of reverse transcription with Superscript II RT (Invitrogen), labelled with ³³P according to the producer's instructions, and hydrolyzed.

For the hybridization membrane filters of the Deutschen Ressourcenzentrum für Genomforschung GmbH (a German center for genome research) (RZPD) were used. This membrane filter was loaded with about 70,000 human cDNA-molecules.

The prepared and labelled samples were hybridized with the membrane filter according to the RZPD's instructions and subsequently washed. The radioactive signals were analyzed after 24 hours of exposition in a phosphorimager.

Analysis

The analysis of the gene expression data from the radioactively labelled filters bases on the measurement of the dye intensities in the digitalized picture. This is achieved by the definition of circular areas over all 57600 spot positions, in which the pixel intensities are integrated. The areas are automatically positioned as accurately as possible over the spots by means of the analysis software (AIDA Array Evaluation, raytest Isotopenmessgeräte GmbH).

In addition to the desired information, namely the amount of nucleic acids bound, contain the detected signals also background signals which are caused by unspecific bindings to the surface of the membrane. In order to eliminate these influences, the background signals are determined in 4608 empty areas of the filter and subtracted as background noise from the hybridization signals.

In order to render the values of different filters comparable, it is necessary to normalize the data afterwards. Due to the high number of spots on the filter, the mean value of all expression values is set as normalization reference. Further, it is necessary to exclude minor spot signals (lower than 10% of the average expression signal), as these are subject to a percentually high error, and would lead to considerable variations of the results when used later on for calculations.

The selection of the genes relevant to SIRS bases on the comparison of the gene expression values in a control person not suffering from SIRS compared to the patient suffering from SIRS. The criteria for the grading of the examined genes is the level of the expression ratio. When comparing the genes of the patients with those of the control, the genes, that were significantly overexpressed or underexpressed, respectively, are the interesting ones.

Table 5 shows that there were 24 genes found in the patient sample, which were significantly overexpressed, if compared with the control sample. Table 6 shows that there were 24 genes found in the patient sample, which were significantly underexpressed, if compared with the control sample. Those results show that the genes listed in table 5 and table 6 correlate with the occurrence of SIRS. Thus, the genes mentioned are leading genes for the diagnosis of SIRS.

TABLE 5
Significantly increased transcription activities and
their relative ratio to the control sample in SIRS

| GenBank | | | | | SEQUENCE- |
|---|---|---|---|---|---|
| Accession No. | HUGO Name | Patient 1: | Patient 2: | Patient 3: | ID |
| R33626 | TFAP2A | 57.57 | 30.43 | 96.57 | 6507 |
| N54839 | CRSP3 | 47.17 | 29.00 | 63.17 | 6552 |
| AA010908 | LCAT | 32.90 | 15.00 | 18.60 | 6561 |
| R59573 | TU12B1 | 85.50 | 60.50 | 49.00 | 6570 |
| R65820 | GEF | 38.00 | 45.80 | 78.00 | 6594 |
| N30458 | NCL | 26.57 | 20.00 | 17.86 | 6624 |
| H86783 | RINZF | 43.33 | 17.00 | 31.33 | 6646 |
| R11676 | CDC20 | 30.75 | 52.00 | 55.25 | 6672 |
| H79834 | SLC20A2 | 16.56 | 14.33 | 27.44 | 6681 |
| H05746 | SLC12A5 | 70.78 | 20.00 | 17.22 | 6685 |
| N21112 | ARHGEF12 | 62.00 | 14.50 | 27.00 | 6693 |
| R71085 | PCANAP7 | 23.00 | 17.63 | 21.96 | 6697 |
| R40287 | NIN283 | 35.00 | 28.00 | 28.00 | 6703 |
| H52708 | PDE2A | 32.78 | 14.11 | 59.22 | 6723 |
| AF086381 | GNPAT | 18.94 | 19.75 | 25.63 | 6725 |
| W57892 | FN1 | 23.61 | 14.67 | 17.06 | 6753 |
| H75516 | KIN | 19.23 | 17.15 | 20.00 | 6761 |
| R59212 | MN1 | 19.65 | 16.65 | 18.61 | 6776 |
| H62284 | CMAH | 23.40 | 36.20 | 32.40 | 6793 |
| W16423 | GCMB | 23.83 | 45.67 | 21.00 | 6818 |
| N40557 | U5 | 55.78 | 20.67 | 22.11 | 6826 |
| H52695 | DDC | 14.80 | 13.70 | 22.30 | 6844 |
| R68244 | HMG14 | 15.81 | 23.19 | 27.56 | 6865 |
| R34679 | ITGB8 | 19.20 | 32.00 | 79.20 | 6874 |

TABLE 6
Significantly reduced transcription activities and
their relative ratio to the control sample in SIRS

| GenBank | | | | | SEQUENCE- |
|---|---|---|---|---|---|
| Accession No. | HUGO Name | Patient 1: | Patient 2: | Patient 3: | ID |
| H18595 | RPL10A | 0.03 | 0.07 | 0.15 | 6553 |
| N90220 | DGUOK | 0.04 | 0.07 | 0.12 | 6574 |
| R19651 | H19 | 0.09 | 0.07 | 0.19 | 6701 |
| R52108 | UBE2D2 | 0.13 | 0.07 | 0.02 | 6741 |
| R83836 | LYN | 0.07 | 0.03 | 0.18 | 6759 |
| H04648 | CSF2RB | 0.06 | 0.07 | 0.13 | 6767 |
| H27730 | PPP2R1B | 0.09 | 0.07 | 0.16 | 6788 |
| N70020 | PRO2822 | 0.10 | 0.04 | 0.11 | 6794 |
| N52437 | CHI3L2 | 0.07 | 0.08 | 0.16 | 6812 |
| W96179 | GCLM | 0.04 | 0.01 | 0.19 | 6822 |
| H42506 | GABARAP | 0.08 | 0.03 | 0.17 | 6842 |
| H66258 | SCP2 | 0.10 | 0.05 | 0.21 | 6846 |
| N38985 | RAP140 | 0.10 | 0.06 | 0.21 | 6896 |
| N73912 | TMP21 | 0.09 | 0.07 | 0.08 | 6905 |
| N51024 | TEGT | 0.08 | 0.06 | 0.07 | 6909 |
| R99466 | EEF1A1 | 0.07 | 0.02 | 0.14 | 7008 |
| R14080 | CAMLG | 0.11 | 0.02 | 0.18 | 7034 |
| W93782 | XPC | 0.12 | 0.05 | 0.21 | 7036 |
| N91584 | RPS6 | 0.06 | 0.05 | 0.12 | 7353 |
| W52982 | PIG7 | 0.05 | 0.07 | 0.10 | 7412 |
| AA033725 | KLF8 | 0.06 | 0.08 | 0.19 | 7535 |
| N20406 | SRP14 | 0.10 | 0.04 | 0.16 | 7565 |
| T83104 | TAF2F | 0.02 | 0.05 | 0.12 | 7630 |
| H79277 | CASP8 | 0.12 | 0.06 | 0.13 | 7677 |

These characteristic changes can be used for the method according to the present invention.

In the appended sequence listing (SEQ. ID No: 6373 to SEQ. ID No: 10540), which is part of the present invention, the gene bank accession numbers indicated in tables 5 and 6 (access via internet via http://www.ncbi.nlm.nih.gov/) of the individual sequences are each allocated to one sequence ID.

### Embodiment 3

### Sepsis

Study of the gene expression of one patient suffering from an early sepsis and one control sample.

The gene expression of one case of an early sepsis and one control sample were measured. The patient's data are summarized in table 7.

TABLE 7
Data of the samples of patients and controls

| | | | | | | | | Apache | | |
|---|---|---|---|---|---|---|---|---|---|---|
| | Gen- | Age | Weight/ | | Intercurrent | | | Score | SAPS II | Selection of |
| | der | [a] | Height | Main diagnosis | diagnosis | Operations | Indication | [point] | [point] | clinical data |
| Patient | male | 70 | 78 kg/ | septic shock | intestine-, | 1. Anastomotic- | Sepsis/ | 19 | 64 | temperature: 35.2° C. |
| | | | 178 cm | after caecum | instable | and sigma re- | septic | | | heart rate: 97/min |
| | | | | perforation and | sternum | resection, rectum | shock | | | MAP 1: 62 mmHg; |
| | | | | post operative | | dead end | | | | art. PH: 7.29 |
| | | | | anastomotic leak | | blockage | | | | Na: 135 mmol/l; |
| | | | | | | 2. Punctation | | | | Creatine: 757 mmol/l; |
| | | | | | | tracheotomy | | | | Cholesterol: - |
| | | | | | | (Griggs) | | | | Breathing rate: 16/min |
| | | | | | | 3. re-wiring | | | | Syst. BP: 105 mmHg; |
| | | | | | | 4. subtotal | | | | Haematocrit: 33% |
| | | | | | | hemiclolectomy | | | | Total number of |
| | | | | | | right side | | | | leucocytes: 13100 |
| | | | | | | 5. definitive | | | | Urea: 19 mmol/l; |
| | | | | | | ileostomy | | | | Diast. BP: 40 mmHg; |
| | | | | | | surgery | | | | PaO2: 12.3 kPa; |
| | | | | | | | | | | K: 4.2 mmol/l; |
| | | | | | | | | | | Bilirubin: 15.1 mmol/l; |
| Control | male | 35 | 90 kg/ | Fracture of the | small hygroma | 1. Craniotomy | Intacranial | 1 | 12 | Temp: 38.8° C. |
| | | | 180 cm | skull, scalp | on both sides | and definite | bleeding | | | heart rate: 84/min |
| | | | | haematoma | | haemostasis | | | | MAP 1: 72 mmHg; |
| | | | | | | | | | | art. PH: 7.42/l |
| | | | | | | | | | | Na: 140 mmol |
| | | | | | | | | | | Creatine: 56 μmol/l; |
| | | | | | | | | | | Breathing rate: 13/min |
| | | | | | | | | | | Syst. BD: 107 mmHg; |
| | | | | | | | | | | Haematocrit: 37% |
| | | | | | | | | | | HCO3: 28.2 mmol/l; |
| | | | | | | | | | | Total number of |
| | | | | | | | | | | leucocytes: 12600 |
| | | | | | | | | | | Urea: 4.7 mmol/l; |
| | | | | | | | | | | Diast. Syst. BD: 54 |
| | | | | | | | | | | mmHg; |
| | | | | | | | | | | PaO2: 10.9 kPa; |
| | | | | | | | | | | K: 3.8 mmol/l; |
| | | | | | | | | | | Bilirubin: 13.4 mmol/l; |

After the whole blood had been drawn, the total RNA was isolated using RNAeasy according to the producer's (Quiagen) instructions. Subsequently, the cDNA was synthesized from the total RNA by means of reverse transcriptions with Superscript II RT (Invitrogen), labelled with ³³P, according to the producer's instructions, and hydrolyzed.

For the hybridization membrane filters of the Deutschen Ressourcenzentrum für Genomforschung GmbH (RZPD) were used. This membrane filter was loaded with about 70,000 humane cDNA-molecules.

The prepared and labelled samples were hybridized by means of the membrane filter according to the RZPD's instructions and subsequently washed. The radioactive signals were analyzed after 24 hours of exposition in a phosphorimager.

The expression ratios of the samples of the patients and the control were calculated from the signal intensities using the AIDA Array Evaluation software.

Table 8 shows that 230 genes of the patient sample were found, which were significantly overexpressed (expression ratios between 13.67 and 98.33), if compared with the control sample. Table 3 further shows that 206 genes of the patient sample were found, which were significantly underexpressed (expression ratios between 0.01 and 0.09), if compared with the control sample. Those results show that the genes listed in table 2 and table 3 correlate with the occurrence of SIRS. Thus, the genes mentioned are leading genes for the diagnosis of an early sepsis.

TABLE 8
Expression ratio of overexpressed genes
of samples of patients and controls

| GenBank | | | |
|---|---|---|---|
| Gene Bank | | Expression ratio of | |
| Accession | | overexpressed genes | SEQUENCE- |
| No. | HUGO Name | compared to control | ID |
| | FLJ20623 | 90.13 | 325 |
| AI272878 | FGF20 | 73.48 | 268 |
| AI218453 | FLJ22419 | 48.8 | 294 |
| AI473374 | SPAM1 | 42.63 | 235 |
| AI301232 | PRG4 | 36.79 | 262 |
| AI452559 | FLJ13710 | 32 | 240 |
| AI339669 | FLJ21458 | 31 | 248 |
| AI142427 | CGRP-RCP | 30 | 331 |
| AA505969 | LOC56994 | 26.67 | 486 |
| AI333774 | AGM1 | 26.19 | 251 |
| W86875 | PSEN1 | 25.66 | 903 |
| AI591043 | NR2E3 | 25 | 196 |
| AI128812 | RBM9 | 23.56 | 324 |
| AA453019 | FLJ21924 | 23.07 | 672 |
| AI690321 | KCNK15 | 22.71 | 134 |
| AA918208 | ADAM5 | 21.83 | 363 |
| AI344681 | ABCA1 | 21.42 | 259 |
| AI654100 | KIAA0610 | 21.04 | 168 |
| AI086719 | FLJ12604 | 20.95 | 326 |
| AA453038 | LOC63928 | 20.74 | 671 |
| AI740697 | SP3 | 20.5 | 114 |
| AI332438 | KIAA1033 | 20.17 | 253 |
| AI734941 | MSR1 | 19.93 | 116 |
| AA541644 | PRV1 | 19.51 | 489 |
| AA513806 | C5ORF3 | 19.3 | 485 |
| AI381513 | B4GALT7 | 18.81 | 273 |
| AI671360 | SIM1 | 18.55 | 154 |
| AI624830 | SAGE | 17.54 | 187 |
| AI001846 | KIAA0480 | 17.54 | 358 |
| AA504336 | TRAP95 | 17.25 | 495 |
| AI142901 | IMPACT | 17.15 | 330 |
| AI077481 | SEMA5B | 17.13 | 327 |
| H41851 | TNFRSF12 | 17.05 | 1511 |
| AI160574 | FLJ23231 | 17 | 314 |
| AI033829 | KIF13B | 16.59 | 339 |
| AI554655 | HLALS | 16.59 | 219 |
| AI074113 | LOC51095 | 16.4 | 328 |
| AA992716 | KIAA1377 | 16.14 | 348 |
| AI382219 | SETBP1 | 16.08 | 272 |
| AI469528 | KIAA1517 | 15.89 | 232 |
| AI090008 | NFYB | 15.76 | 349 |
| AI203498 | WRN | 15.72 | 310 |
| AI832179 | HPGD | 15.66 | 65 |
| AI278521 | SPRR3 | 15.61 | 265 |
| AA909201 | FLJ23129 | 15.12 | 361 |
| AI383932 | ZNF214 | 14.98 | 269 |
| AA455096 | MDM1 | 14.9 | 652 |
| AA953859 | NOL4 | 14.68 | 363 |
| R56800 | GDF1 | 14.67 | 1755 |
| AI676097 | FCER1A | 14.54 | 151 |
| AI380703 | KIAA1268 | 14.51 | 275 |
| AI832086 | RTKN | 14.51 | 66 |
| AI125328 | FLJ22490 | 14.33 | 317 |
| AI056693 | LOC57115 | 14.3 | 329 |

TABLE 9
Expression ratio of underexpressed genes
of samples of patients and controls

| GenBank | | Expression ratio of | |
|---|---|---|---|
| Accession | | underexpressed genes | SEQUENCE- |
| No. | Hugo Name | compared to control | ID |
| R15296 | C9ORF9 | 0.01 | 2050 |
| AA609149 | FLJ10058 | 0.01 | 375 |
| AI566451 | KAI1 | 0.01 | 211 |
| AI334246 | PDCD7 | 0.01 | 250 |
| H38679 | NXPH3 | 0.01 | 1477 |
| AI696866 | KIAA1430 | 0.01 | 130 |
| AI922915 | FLJ00012 | 0.01 | 23 |
| AI889612 | KPNA6 | 0.01 | 46 |
| AI921695 | FLJ23556 | 0.02 | 26 |
| AA410933 | HRH1 | 0.02 | 764 |
| AA705423 | LOC57799 | 0.02 | 383 |
| AI206507 | RAD54B | 0.02 | 298 |
| AI921327 | MED6 | 0.02 | 28 |
| AI682701 | VNN1 | 0.02 | 146 |
| H82822 | METAP2 | 0.02 | 1352 |
| AI890612 | MAGE1 | 0.02 | 42 |
| AI262169 | ALDOB | 0.02 | 257 |
| H44908 | C21ORF51 | 0.02 | 1502 |
| AI572407 | FLJ22833 | 0.02 | 203 |
| AI924869 | STX4A | 0.02 | 19 |
| AI925556 | AF140225 | 0.02 | 12 |
| AI798388 | KIAA0912 | 0.03 | 95 |
| AI623978 | SCEL | 0.03 | 188 |
| AI889598 | MLYCD | 0.03 | 47 |
| AI889648 | PAWR | 0.03 | 45 |
| AI431323 | AREG | 0.03 | 237 |
| AA446611 | CDH6 | 0.03 | 706 |
| AI697365 | P53DINP1 | 0.03 | 129 |
| H82767 | VAMP3 | 0.03 | 1353 |
| AI688916 | FLJ10933 | 0.03 | 137 |
| AI888660 | FLJ11506 | 0.03 | 51 |
| AI890314 | RAB6B | 0.03 | 43 |
| AI653893 | LAMA5 | 0.03 | 169 |
| R89811 | HGFAC | 0.03 | 1462 |
| AI863022 | MAGEA4 | 0.04 | 59 |
| AA749151 | XPOT | 0.04 | 378 |
| AI355007 | ITPKB | 0.04 | 246 |
| AI582909 | MESDC2 | 0.04 | 201 |
| AI832016 | APOL1 | 0.04 | 67 |
| H11827 | THOP1 | 0.04 | 1597 |
| AI560205 | KIAA1841 | 0.04 | 216 |
| AA503092 | UMPH1 | 0.04 | 490 |
| AI932616 | FLJ22294 | 0.04 | 5 |
| AI799137 | FLJ11274 | 0.04 | 93 |
| AI686838 | SARDH | 0.04 | 142 |
| AI623132 | SREC | 0.04 | 189 |
| R96713 | DKFZP434A0131 | 0.04 | 1442 |
| AI674926 | LBC | 0.04 | 152 |
| AI886302 | HRI | 0.04 | 53 |
| AI434650 | MGC2560 | 0.04 | 238 |
| AI631380 | GNG4 | 0.04 | 180 |
| AA508868 | ORC6L | 0.04 | 491 |
| AI620374 | HP1-BP74 | 0.04 | 190 |
| AI679115 | KIAA1353 | 0.04 | 148 |
| AA652703 | MRPL49 | 0.04 | 386 |
| AI355775 | CDK3 | 0.04 | 245 |

These characteristic alterations can be used in particular for the method of the present invention.

In the appended sequence listing, which is part of the present invention, the gene bank accession numbers (access via internet via http://www.ncbi.nlm.nih.gov/) indicated in tables 8 and 9 of the individual sequences are each allocated to one sequence ID.

Implementation:

Preparation of RNA. The conditioned media were removed from the culture flasks and the adherent cells were lysed directly in the culture flasks using TRIzol-reagent (GIBCO/BRL) according to the producer's instructions. One deproteinization cycle was carried out and afterwards, the RNA was precipitated by adding isopropyl alcohol, afterwards rinsed with ethyl alcohol, and again solved in 200 μl RNA-save resuspension solution (Ambion, Austin, Tex.). The RNA preparations were degraded with 0.1 units/μl DNase I, in DNase 1 buffer from CLONTECH. Additionally, proteins were removed from the RNA units in an alcohol mixture comprising phenol, chloroform and isoamyl alcohol, precipitated by adding ethyl alcohol, and solved in 50-100 μl RNA-save resuspension solution. The RNA concentration was spectro-photometrically determined, provided that 1A₂₆₀ corresponds to a concentration of 40 μg/ml. The samples were adapted to a final concentration of 1 mg/ml und stored at 80° C. No signs of deterioration of quality were observed. By means of agarose electrophoresis it was evaluated whether the RNA preparations were complete (i.e. they were not decayed into their components), here, RNA-standards (GIBCO/BRL) were used. Each of the preparations described herein contained intact RNA the 28S-, 18S- and 5S-bands of which were clearly detectable (data not given). No recognizable differences between healthy and infectious cells were determined with regard to the electrophoretically determined RNA samples.

Preparation of radioactively labelled cDNA-samples and hybridizing by means of DNA arrays. The cDNA-synthesis was carried out according to the producer's instructions using gene specific primer (CLONTECH) and [³²P]-dATP with Moloney Murine Leukemea Virus Reverse Transkriptase (SuperScript II, GIBCO/BRL). For the cDNA-synthesis, the same amounts of RNA (5 μg) were used from each sample.

Alternative

RNA was extracted from the tissue samples by means of guanidinium thiocyanate and afterwards centrifuged in CsCl as described [19]. The RNA was extracted according to the producer's instructions from the cell lines with RNAzol (Biotex Laboratories, Houston). The poly(A) RNA was isolated from 500 μg RNA by means of DynaBeads (Dynal, Oslo), as described in the producer's recommendations.

The differences in the gene expression were examined using Atlas Array membranes (CLONTECH). A first short step was the transcription of 1 μg RNA of each cell line in [−³²P]dATP-labelled cDNA at a time.

Analysis

The analysis of the gene expression data from the radioactively labelled filters bases on the measurement of the dye intensities in the digitalized picture. This is achieved by the definition of circular areas over all 57600 spot positions, in which the pixel intensities are integrated. The areas are automatically positioned as accurately as possible over the spots by means of the analysis software (AIDA Array Evaluation, raytest Isotopenmessgeräte GmbH).

In addition to the desired information, namely the amount of nucleic acids bound, contain the detected signals also background signals which are caused by unspecific bindings to the surface of the membrane. In order to eliminate these influences, the background signals are determined in 4608 empty areas of the filters and subtracted as background noise from the hybridization signals.

It is possible to distinguish between punctual signals that are caused on the filter by dust particles or other disturbances instead of binding of nucleic acids, and real spots, due to their irregular form, and the punctual signals are excepted from further analysis.

In order to render the values of different filters comparable, it was necessary to normalize the data afterwards. Due to the high number of spots on the filter, the mean value of all expression values is set as normalization reference. Further, it is necessary to exclude minor spot signals (lower than 10% of the average expression signal), as these are subject to a percentually high error, and would lead to considerable variations of the results when used later on for calculations.

The selection of the genes relevant to SIRS/sepsis bases on the comparison of the gene expression values in a control person without SIRS/sepsis compared to one patient at a time suffering from sepsis/SIRS. The criteria for the grading of the examined genes is the level of the expression ratio. The interesting genes are those which were most overexpressed or underexpressed, respectively, in the patients compared with the control.

### Embodiment 4

### Sepsis

Study of the protein expression of one patient suffering from sepsis and one control sample.

The protein expression of one case of sepsis and one control sample were measured. The patients' data are summarized in table 10.

TABLE 10
Data of the samples of patients and controls

| | | Age | | Main | |
|---|---|---|---|---|---|
| | Gender | [a] | Weight/Height | diagnosis | Intercurrent diagnosis |
| Control | female | 21 | 62 kg/167 cm | cranio- | Generalized cerebral oedema, brain stem contusion, |
| | | | | cerebral- | fracture of the lateral orbital pillar, fracture |
| | | | | trauma | wall left side, lateral fracture of the nasal |
| | | | | | sceleton, bleeding into the right side ventricle, |
| | | | | | free air intracraniellfrontally left side, ethmoid |
| | | | | | bone fracture, fracture of the front pelvic ring with |
| | | | | | impression and dislocation of the fragments, fracture |
| | | | | | of the massa lateralis of the OS sacrum right side |
| | | | | | in the heigh of S1/S2, clavikular fracture left side |
| Patient | male | 59 | 70 kg/175 cm | septic shock | pleural effusions on both sides, multi organ failure, |
| | | | | after | multiple necrosis of the acra and pretibial on both |
| | | | | perforation of | sides, arterial microembolism, arterial thrombosis, |
| | | | | one ulcus | secundary thrombocytopenia, acute kidney failure |
| | | | | pylori and | |
| | | | | subsequent 4 | |
| | | | | quadrant | |
| | | | | peritonitis | |

| | | | Apache | | |
|---|---|---|---|---|---|
| | | | Score | SAPS II | |
| | Operations | Indication | [point] | [point] | Selection of clinical data |
| Control | none | not applicable | 21 | — | temperature: 35.3° c. |
| | | | | | heart rate: 146/min |
| | | | | | map 1: 68 mmhg; art. ph: 7.48 |
| | | | | | na: 145 mmol/l; ceratine: 52 μmol/l; |
| | | | | | syst. bp: 94 mmhg; diast. bp: 56 mmhg; |
| | | | | | haematocrit: 0.26% |
| | | | | | total number of leucocytes: 9200 |
| | | | | | urea: 7.1 mmol/l; |
| | | | | | k: 5 mmol/l; |
| | | | | | bilirubin: 11.1 mmol/l; |
| Patient | relaparotomy, | septic shock | 28 | 74 | temperature: 37.7° c. |
| | lavage, and partial | | | | heart rate: 139/min |
| | resection of the | | | | map 1: 64 mmhg; art. ph: 7.15 |
| | omentum | | | | na: 142 mmol/l, ceratine: 187 mmol/l; |
| | | | | | breathing rate: 19/min |
| | | | | | syst. bp: 99 mmhg; diast. bp: 49 |
| | | | | | mmhg; haematocrit: 24% |
| | | | | | hco3: 13.7 mmol/l, total number of |
| | | | | | leucocytes: 5200 |
| | | | | | urea: 27.6 mmol/l; |
| | | | | | pao2!: 13.2 kpa, k: 5.3 mmol/l; |
| | | | | | bilirubin: 33.9 mmol/l; |
| | | | | | urine: 110 ml, 14 h |

Whole blood was drawn and inserted into a serum tube and centrifugation (5500 rcf; 10 min; 4° C.) was carried out. The supernatant of serum was transferred into cryo tubes immediately upon centrifugation and stored at −35° C.

To downgrade the albumin, the serum was treated with Affi-Gel Blue Affinity Chromatography Gel for Enzyme and Blood Protein Purifications (Bio-Rad) according to the producer's instructions. To avoid undesired interactions of protein and matrix, the equilibration- and binding buffer were added 400 mM NaCl.

Non-binding proteins were collected and precipitated with methanol and chloroform according to the protocol of Wessel and Flügge (Anal. Biochem. 1984 April; 138(1): 141-3). 250 microgram of precipitated serum protein were added to a solution consisting of 8M urea; 2.0 M thiourea; 4% CHAPS; 65 mM DTT and 0.4% (w/v) Bio-Lytes 3/10 (Bio-Rad) and subjected to an isoelectric focusing as well as a subsequent SDS-PAGE.

### SDS-PAGE

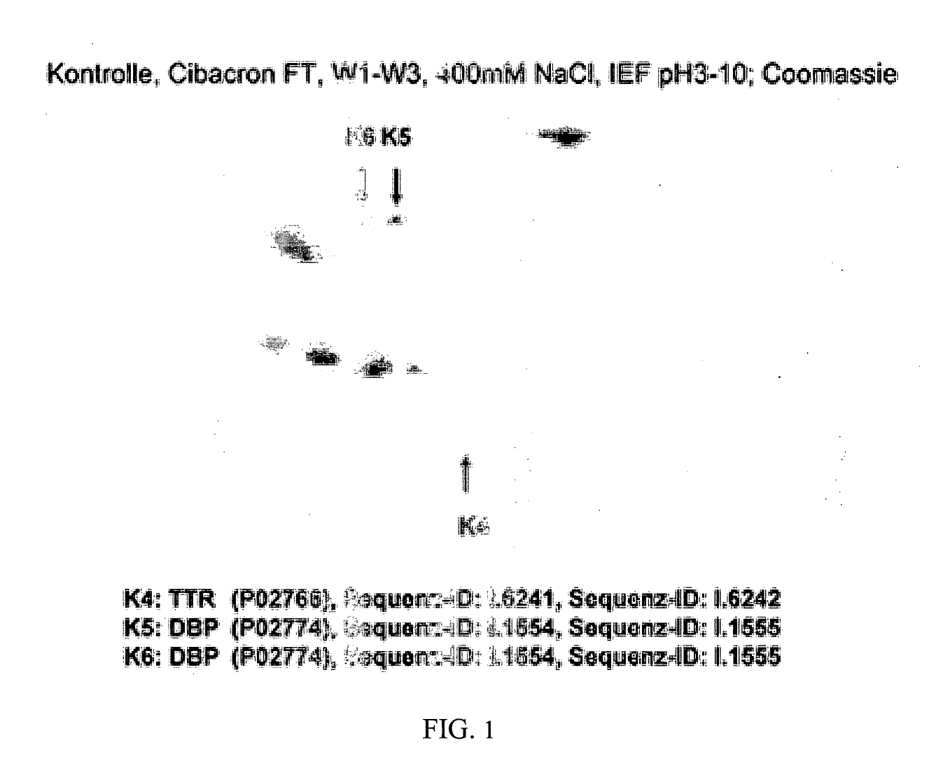

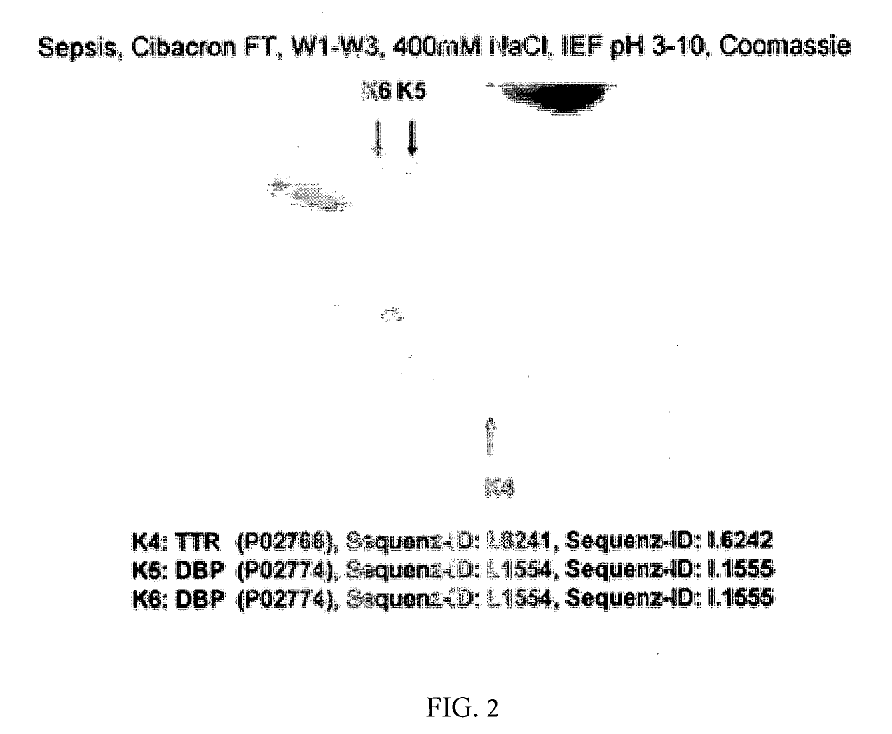

K4 in FIG. 1 and in FIG. 2 is the acute phase protein transthyretin (TTR; P02766, SEQ. ID 6241, SEQ. ID 6242) and K5 and K6 are the vitamin D-binding protein (DBP; P02774, SEQ. ID 1554, SEQ. ID 1555).

The gels can be produced as follows (Cibacron FT, W1-W3, 400 mM NaCl, IEF pH 3-10, Coomassie):

250 microgram of precipitated serum protein were added to a solution consisting of 8M urea; 2.0 M thiourea; 4% CHAPS; 65 mM DTT and 0.4% (w/v) Bio-Lytes 3/10 (Bio-Rad) and subjected to an isoelectric focusing as well as a subsequent SDS-PAGE.

The prepared 2-dimensional gels were colored with Coomassie Brilliant Blau G-250 and differentially expressed proteins were identified by mass spectroscopy.

A comparing analysis shows (FIG. 1, FIG. 2=that the acute phase protein transthyretin (TTR; P02766, SEQ. ID: 6241, SEQ. ID 6242), as well as the vitamin D-binding protein (DBP; P02774, SEQ. ID 1554, SEQ. ID 1555) are less expressed by the sepsis patient, if compared with the control patient.

These results clearly indicate that the protein expression or the protein composition, respectively, of serum and plasma change in the course of the disease.

### Embodiment 5

### Severe Sepsis

Studies of differential gene expression with patients suffering from severe sepsis.

Whole blood samples of patients who were under the care of a surgical intensive care unit were examined for the measurement of the differential gene expression in connection with severe sepsis.

Control samples were whole blood samples of the patients that were drawn after an uncomplicated neurosurgical operation. The patients were treated on the same intensive care unit. No one of these patients developed an infection and/or showed clinical signs of a generalized inflammatory reaction (defined according to the SIRS-criteria [4]) during the whole time of stationary treatment.

Additionally, whole blood samples were drawn from six male and two female patients (patients' samples). In the time period of 24 hours before the whole blood was drawn, each of these patients developed a new severe sepsis with organ dysfunction. A range of characteristics of the patients suffering from severe sepsis are shown in table 1. Information concerning the age, gender, the cause of the severe sepsis (see diagnosis) as well as concerning the clinical severity, measured with the APACHE-II- and SOFA-Scores (in points each), that are well documented in clinical literature, is given. Equally, the plasma protein levels of procalcitonin (PCT), a new kind of sepsis label, are given, as well as the individual survival conditions.

TABLE 11
Data of the group of patients

| | | | | Apache II | SOFA | | |
|---|---|---|---|---|---|---|---|
| | | | Classification | Score | Score | PCT | survival |
| Age | Gender | Diagnosis | according to [4] | [points] | [points] | [ng/ml] | conditions |
| 68 | female | peritonitis | severe | 17 | 4 | 269 | died |
| | | | sepsis/ | | | | |
| 39 | male | ARDS | septic shock | 17 | 11 | 0.39 | died |
| 36 | male | peritonitis | septic shock | 11 | 7 | 9.77 | survived |
| 80 | male | peritonitis | severe | 28 | 4 | 23.61 | survived |
| | | | sepsis | | | | |
| 32 | male | bacterial | septic shock | 21 | 7 | 1.69 | survived |
| | | pancreatitis | | | | | |
| 73 | male | ARDS | septic shock | 16 | 14 | 9.96 | died |
| 67 | male | ARDS | septic shock | 24 | 12 | 12.88 | survived |
| 76 | female | peritonitis | septic shock | 30 | 11 | 4.19 | died |

After the whole blood had been drawn, the total RNA was isolated using the PAXGene Blood RNS Kit according to the producer's (Qiagen) instructions. Subsequently, the cDNA was synthesized from the total RNA by means of reverse transcription with Superscript II RT (Invitrogen) according to the producer's instructions, labelled with aminoallyl-dUTP and succinimidylester of the fluorescent dyes Cy3 and Cy5 (Amersham), and hydrolyzed.

The microarrays (Lab-Arraytor human 500-1 cDNA) of the company SIRS-Lab GmbH were used for the hybridization. These micorarrays are loaded with 340 human cDNA-molecules. The 340 human cDNA-molecules are 3-fold immobilized in three subarrays on each microarray.

The prepared and labelled samples were hybridized with the microarrays according to the producer's instructions and subsequently washed. The fluorescence signals of the hybridized molecules were measured by means of a scanner (AXON 4000B).

Analysis

One test was analyzed by means of scanned pictures of the microarrays after hybridization. The mean intensity value of the detected spots were defined as the measured expression value of the corresponding gene. Spots were automatically identified by means of picture analysis and their homogeneity was checked. The analysis was controlled manually. The detected signals comprise not only the desired information, namely the amount of nucleic acids bound, but also background signals which are caused by unspecific bindings to the surface of the membrane. The definition of the signals of the background rendered an optimum differentiation between spots and the surface of the chip possible, which surface also showed color effects. For the analysis of the microarrays blank spots were chosen as background. The mean expression value of the chosen blank spots within one block (of 14 times 14 spots) was subtracted from the expression values of the gene spots (in the corresponding block).

It was possible to distinguish between punctual signals that were caused on the filter by dust particles or other disturbances instead of bindings of nucleic acids, and real spots, due to their irregular form, and the punctual signals were excepted from further analysis.

In order to render the values between the 3 subarrays and between different microarrays comparable, it was necessary to normalize the data afterwards. Due to the high number of spots on the microarray, the mean value of all expression values was set as normalization reference. The mean expression per gene was calculated by choosing the two (from three) repetitions that were closest to each other.

The expression ratios of the samples of the patients and the control were calculated from the signal intensities using the AIDA Array Evaluation software. The criterion for the grading of the examined genes was the level of the expression ratio. The interesting genes were those which were most overexpressed or underexpressed, respectively, compared with the control samples.

Table 12 shows that 41 genes of the patient sample were found, which were significantly overexpressed, if compared with the control sample. Table 13 shows that 89 genes of the patient sample were found, which were significantly underexpressed, if compared with the control sample. Those results show that the genes listed in table 12 and table 13 correlate with the occurrence of a severe sepsis. Furthermore, these results correlate with the clinical classification according to [4] as well as patients' PCT-concentrations, that are typical for the occurrence of a severe sepsis [23]. Thus, the gene activities of the genes mentioned are labels for the diagnosis of a severe sepsis.

TABLE 12
Expression ratio of overexpressed genes
of samples of patients and controls

| GenBank | | Expression ratio of | |
|---|---|---|---|
| Accession | | overexpressed genes | SEQUENCE- |
| No. | HUGO Name | compared to control | ID |
| XM_086400 | S100A8 | 4.4 | 6243 |
| XM_001682 | S100A12 | 3.03 | 6244 |
| NM_002619 | PF4 | 2.21 | 6245 |
| NM_002704 | PPBP | 1.66 | 6246 |
| NM_001101 | ACTB | 1.65 | 6247 |
| NM_001013 | RPS9 | 1.61 | 6248 |
| XM_057445 | SELP | 1.61 | 6249 |
| BC018761 | IGKC | 1.53 | 6250 |
| XM_030906 | TGFB1 | 1.51 | 6251 |
| NM_001760 | CCND3 | 1.48 | 6252 |
| XM_035922 | IL11 | 1.28 | 6253 |
| XM_039625 | DUSP10 | 1.17 | 6254 |
| XM_002762 | TNFAIP6 | 1.17 | 6255 |
| XM_015396 | ALOX5AP | 1.15 | 6256 |
| NM_003823 | TNFRSF6B | 1.15 | 6257 |
| XM_029300 | DPP4 | 1.15 | 6258 |
| NM_001562 | IL18 | 1.14 | 6259 |
| NM_005037 | PPARG | 1.11 | 6260 |
| M90746 | FCGR3B | 1.07 | 6261 |
| NM_001315 | MAPK14 | 0.99 | 6262 |
| BC001506 | CD59 | 0.88 | 6263 |
| XM_042018 | BSG | 0.88 | 6264 |
| XM_010177 | DUSP9 | 0.87 | 6265 |
| BC013992 | MAPK3 | 0.84 | 6266 |
| NM_001560 | IL13RA1 | 0.82 | 6267 |
| NM_004555 | NFATC3 | 0.74 | 6268 |
| NM_001154 | ANXA5 | 0.73 | 6269 |
| NM_001310 | CREBL2 | 0.7 | 6270 |
| XM_036107 | ITGB2 | 0.65 | 6271 |
| XM_009064 | JUNB | 0.65 | 6272 |
| NM_001774 | CD37 | 0.62 | 6273 |
| XM_049849 | TNFRSF14 | 0.6 | 6274 |
| NM_003327 | TNFRSF4 | 0.57 | 6275 |
| BC001374 | CD151 | 0.56 | 6276 |
| XM_051958 | ALOX5 | 0.56 | 6277 |
| NM_021805 | SIGIRR | 0.5 | 6278 |
| NM_017526 | HSOBRGR | 0.48 | 6279 |
| XM_011780 | DAPK1 | 0.46 | 6280 |
| NM_006017 | PROML1 | 0.44 | 6281 |
| D49410 | IL3RA | 0.43 | 6372 |
| XM_027885 | RPL13A | 0.33 | 6282 |

TABLE 13
Expression ratio of underexpressed genes
of samples of patients and controls

| GenBank | | Expression ratio of | |
|---|---|---|---|
| Accession | | underexpressed genes | SEQUENCE- |
| No. | HUGO Name | compared to control | ID |
| NM_007289 | MME | −2.11 | 6283 |
| XM_008411 | SCYA13 | −2.06 | 6284 |
| XM_055188 | ENG | −2.01 | 6285 |
| NM_021073 | BMP5 | −1.99 | 6286 |
| XM_007417 | TGFB3 | −1.93 | 6287 |
| NM_001495 | GFRA2 | −1.88 | 6288 |
| XM_009475 | AHCY | −1.86 | 6289 |
| XM_006738 | CD36L1 | −1.86 | 6290 |
| NM_001772 | CD33 | −1.86 | 6291 |
| NM_057158 | DUSP4 | −1.83 | 6292 |
| XM_058179 | CD244 | −1.77 | 6293 |
| NM_001770 | CD19 | −1.75 | 6294 |
| NM_004931 | CD8B1 | −1.73 | 6295 |
| XM_006454 | CD3G | −1.71 | 6296 |
| XM_041847 | TNF | −1.65 | 6297 |
| NM_145319 | MAP3K6 | −1.62 | 6298 |
| XM_045985 | ITGA2B | −1.61 | 6299 |
| XM_055756 | TIMP1 | −1.61 | 6300 |
| NM_004740 | TIAF1 | −1.61 | 6301 |
| XM_008432 | ITGA3 | −1.57 | 6302 |
| XM_034770 | PAFAH1B1 | −1.56 | 6303 |
| NM_014326 | DAPK2 | −1.55 | 6304 |
| XM_043864 | PIK3R1 | −1.49 | 6305 |
| U54994 | CCR5 | −1.49 | 6306 |
| NM_004089 | DSIPI | −1.49 | 6307 |
| XM_037260 | F2R | −1.45 | 6308 |
| NM_172217 | IL16 | −1.45 | 6309 |
| AF244129 | LY9 | −1.45 | 6310 |
| NM_003775 | EDG6 | −1.43 | 6311 |
| NM_001781 | CD69 | −1.41 | 6312 |
| NM_019846 | CCL28 | −1.39 | 6313 |
| NM_001511 | CXCL1 | −1.38 | 6314 |
| NM_006505 | PVR | −1.33 | 6315 |
| NM_000075 | CDK4 | −1.33 | 6316 |
| XM_042066 | MAP3K1 | −1.32 | 6317 |
| NM_003242 | TGFBR2 | −1.31 | 6318 |
| NM_003874 | CD84 | −1.31 | 6319 |
| XM_033972 | ATF6 | −1.3 | 6320 |
| XM_001840 | PLA2G2A | −1.3 | 6321 |
| NM_018310 | BRF2 | −1.29 | 6322 |
| AF212365 | IL17BR | −1.25 | 6323 |
| XM_056798 | CD81 | −1.25 | 6324 |
| NM_000061 | BTK | −1.24 | 6325 |
| XM_001472 | JUN | −1.23 | 6326 |
| XM_007258 | TNFAIP2 | −1.23 | 6327 |
| XM_048555 | IFNAR2 | −1.23 | 6328 |
| XM_041060 | FOS | −1.23 | 6329 |
| XM_056556 | TNFSF7 | −1.23 | 6330 |
| XM_016747 | LTBP1 | −1.22 | 6331 |
| XM_006953 | TNFRSF7 | −1.21 | 6332 |
| NM_015927 | TGFB1I1 | −1.19 | 6333 |
| XM_010807 | INHBB | −1.16 | 6334 |
| NM_002184 | IL6ST | −1.14 | 6335 |
| XM_008570 | VAMP2 | −1.13 | 6336 |
| NM_006856 | ATF7 | −1.1 | 6337 |
| NM_000674 | ADORA1 | −1.09 | 6338 |
| NM_000173 | GP1BA | −1.08 | 6339 |
| XM_048068 | SCYD1 | −1.07 | 6340 |
| NM_022162 | CARD15 | −1.07 | 6341 |
| NM_001199 | BMP1 | −1.02 | 6342 |
| NM_000960 | PTGIR | −1.01 | 6343 |
| XM_012039 | FUT4 | −0.99 | 6344 |
| XM_034166 | NOS2A | −0.99 | 6345 |
| NM_003188 | MAP3K7 | −0.98 | 6346 |
| NM_006609 | MAP3K2 | −0.98 | 6347 |
| XM_027358 | PCMT1 | −0.95 | 6348 |
| XM_007189 | FOXO1A | −0.93 | 6349 |
| XM_030523 | MAP3K8 | −0.92 | 6350 |
| XM_002923 | CCR2 | −0.88 | 6351 |
| XM_054837 | TNFRSF1B | −0.87 | 6352 |
| NM_000634 | IL8RA | −0.87 | 6353 |
| NM_004590 | CCL16 | −0.86 | 6354 |
| XM_012717 | CSNK1D | −0.86 | 6355 |
| XM_012649 | SCYA7 | −0.84 | 6356 |
| XM_008679 | TP53 | −0.84 | 6357 |
| XM_030509 | PTGIS | −0.83 | 6358 |
| XM_039086 | CDW52 | −0.82 | 6359 |
| XM_027978 | CFLAR | −0.81 | 6360 |
| NM_005343 | HRAS | −0.79 | 6361 |
| XM_043574 | DAP3 | −0.78 | 6362 |
| NM_002188 | IL13 | −0.77 | 6363 |
| XM_055699 | ENTPD1 | −0.72 | 6364 |
| NM_000565 | IL6RA | −0.67 | 6365 |
| NM_002211 | ITGB1 | −0.65 | 6366 |
| XM_049864 | CSF3 | −0.63 | 6367 |
| XM_045933 | CAMKK2 | −0.63 | 6368 |
| NM_033357 | CASP8 | −0.55 | 6369 |
| XM_008704 | DNAM-1 | −0.52 | 6370 |
| NM_030751 | TCF8 | −0.5 | 6371 |

It is for example possible to take advantage of these characteristic changes in the method of the present invention.

In the appended sequence listing, which is part of the present invention, the gene bank accession numbers (access via internet via http://www.ncbi.nlm.nih.gov/) indicated in tables 12 and 13 of the individual sequences are each allocated to one sequence ID. (SEQ. ID No.: 6243 to SEQ. ID No. 6372). The following sequence listing is part of the present invention.

### REFERENCES

1. Natanson C, Hoffmann W D, Suffredini A, Eichacker P Q, and Danner R L: Selected treatment strategies for septic shock based on proposed mechanisms of pathogenesis. Ann Intern Med 1994; 120:771-783
2. Danner R L, Elin R J, Hosseini J M, Wesley R A, Reilly J M, Parillo J E: Endotoxemia in human septic shock. Chest 1991; 99: 169-175
3. Niederman M S, Fein A M: sepsis syndrome, the adult respiratory distress syndrome, and nosocomial pneumonia. A common clinical sequence. Clin Chest Med 1990; 11: 633-656
4. American College of Chest Physicians/Society of Critical Care Medicine Consensus Conference: definitions for sepsis and organ failure and guidelines for the use of innovative therapies in sepsis. Crit Care Med 1992; 20: 864-874
5. Brun-Buisson C, Doyon F, Carlet J, Dellamonica P, Gouin F, Lepoutre A, Mercier J C, Offenstadt G, Regnier B: Incidence, risk factors, and outcome of severe sepsis and septic shock in adults. A multicenter prospective study in intensive care units. French ICU Group for Severe Sepsis. JAMA 1995; 274: 968-974
6. Le-Gall J R, Lemeshow S, Leleu G, Klar J, Huillard J, Rue M, Teres D, Artigas A: Customized probability models for early severe sepsis in adult intensive care patients. Intensive Care Unit Scoring Group. JAMA 1995; 273: 644-650
7. Increase in National Hospital Discharge Survey rates for septicemia—United States, 1979-1987. MMWR Morb Mortal Wkly Rep 1990; 39: 31-34
8. Pittet D, Tarara D, Wenzel R P: Nosocomial bloodstream infection in critically ill patients. Excess length of stay, extra costs, and attributable mortality. JAMA 1994; 271: 1598-1601
9. Vincent J L, Angus D, Annane D, et al. (2001) Clinical expert round table discussion (session 5) at the Margaux Conference on Critical Illness: outcomes of clinical trials in sepsis: lessons learned. Crit Care Med 29:S136-137.
10. Southern E M (1974) An improved method for transferring nucleotides from electrophoresis strips to thin layers of ion-exchange cellulose. Anal Biochem 62:317-318
11. Gillespie D, Spiegelman S (1965) A quantitative assay for DNA-RNA hybrids with DNA immobilized on a membrane. J Mol Biol 12:829-842
12. Lennon G G, Lehrach H (1991) Hybridization analyses of arrayed cDNA libraries. Trends Genet 7: 314-317
13. Kafatos F C, Jones C W, Efstratiadis A (1979) Determination of nucleic acid sequence homologies and relative concentrations by a dot hybridization procedure. Nucl Acid Res 7:1541-1552
14. Fodor S P, Read J L, Pirrung M C, Stryer L, Lu A T, Solas D (1991) Light-directed, spatially addressable parallel chemical synthesis. Science 251:767-773
15. Pease A C, Solas D, Sullivan E J, Cronin M T, Holmes C P, Fodor SP (1994) Light-generated oligonucleotide arrays for rapid DNA sequence analysis. Proc Natl Acad Sci USA 91:5022-5026
16. Schena M, Shalon D, Davis R W, Brown P O (1995) Quantitative monitoring of gene expression patterns with a complementary DNA microarray. Science 270:467-470
17. Golub T R, Slonim D K, Tamayo P, et al. (1999) Molecular classification of cancer: class discovery and class prediction by gene expression monitoring. Science 286:531-537
18. Alizadeh A A, Eisen M B, Davis R E, et al. (2000) Distinct types of diffuse large B-cell lymphoma identified by gene expression profiling. Nature 403:503-511
19. Varambally S, Dhanasekaran S M, Zhou M, et al. (2002) The polycomb group protein EZH2 is involved in progression of prostate cancer. Nature 419:624-629
20. Fillion I, Ouellet N, Simard M, et al. (2002) Role of chemokines and formyl peptides in pneumococcal pneumonia-induced monocyte/macrophage recruitment. J. Immunol.; 166(12):7353-61.
21. Zhao B, Bowden R A, Stavchansky S A, Bowman P D (2001) Human endothelial cell response to gram-negative lipopolysaccharide assessed with cDNA microarrays. Am J Physiol Cell Physiol. November; 281(5):C1587-95.
22. Chinnaiyan A M, Huber-Lang M, Kumar-Sinha C et al. (2001) Molecular signatures of sepsis: multiorgan gene expression profiles of systemic inflammation. Am J Pathol. 159(4):1199-209.

1. A method for in vitro detection of acute generalized inflammatory conditions (SIRS), comprising:
isolating sample RNA from a sample of a mammal;
labelling of the sample RNA and/or at least one DNA being a gene or gene fragment specific for SIRS, with a detectable label.
contacting the sample RNA with the DNA under hybridization conditions;
contacting sample RNA representing a control for non-pathologic conditions, with at least one DNA, under hybridization conditions, whereby the DNA is a gene or gene fragment specific for SIRS;
quantitative detection of the label signals of the hybridized sample RNA and control RNA; and
comparing the quantitative data of the label signals in order to determine whether the genes or gene fragments specific for SIRS are more expressed in the sample than in the control, or less.

2. A method for in vitro detection of sepsis and/or sepsis-like conditions,
isolating of sample RNA from a sample of a mammal;
labelling of the sample RNA and/or at least one DNA being a gene or gene fragment specific for sepsis, with a detectable label.
contacting the sample RNA with the DNA under hybridization conditions;
contacting sample RNA representing a control for non-pathologic conditions, with at least one DNA, under hybridization conditions, whereby the DNA is a gene or gene fragment specific for sepsis and/or sepsis-like conditions;
quantitative detection of the label signals of the hybridized sample RNA and control RNA; and
comparing the quantitative data of the label signals in order to determine whether the genes or gene fragments specific for sepsis and/or sepsis-like conditions are more expressed in the sample than in the control, or less.

3. A method for in vitro detection of severe sepsis, comprising:
isolating of sample RNA from a sample of a mammal;
labelling of the sample RNA and/or at least one DNA being a gene or gene fragment specific for severe sepsis, with a detectable label.
contacting the sample RNA with the DNA under hybridization conditions;
contacting sample RNA representing a control for non-pathologic conditions, with at least one DNA, under hybridization conditions, whereby the DNA is a gene or gene fragment specific for severe sepsis;
quantitative detection of the label signals of the hybridized sample RNA and control RNA; and
comparing the quantitative data of the label signals in order to determine whether the genes or gene fragments specific for severe sepsis are more expressed in the sample than in the control, or less.

4. The method of claim 1, characterized in that the control RNA is hybridized with the DNA before the measurement of the sample RNA and the label signals of the control RNA/DNA-complex is gathered and, if necessary, recorded in form of a calibration curve or table.

5. The method of claim 1, characterized in that unchanged genes from sample and/or control RNA are used as reference genes for the quantification.

6. The method of claim 1, characterized in that mRNA is used as sample RNA.

7. The method of claim 1, characterized in that the DNA is arranged, particularly immobilized, on predetermined areas on a carrier in the form of a microarray.

8. The method of claim 1, characterized in that the method for early detection by means of differential diagnostics, for control of the clinical and therapeutic progress, for the individual risk evaluation in patients, for the evaluation whether the patient will respond to a specific treatment, as well as for post mortem diagnosis of SIRS and/or sepsis and/or severe sepsis and/or systemic infections and/or septic conditions and/or infections.

9. The method of claim 1, characterized in that the sample is selected from the following group: body fluids, in particular blood, liquor, urine, ascitic fluid, seminal fluid, saliva, puncture fluid, cell content, or a mixture thereof.

10. The method of claim 1, characterized in that cell samples are subjected a lytic treatment, if necessary, in order to free their cell contents.

11. The method of claim 1, characterized in that the mammal is a human.

12. The method of claim 1, characterized in that the gene or gene segment specific for SIRS is selected from the group consisting of SEQUENCE ID No. III.1 to SEQUENCE ID No. III.4168, as well as gene fragments thereof with 5-2000 or more, preferably 20-200, more preferably 20-80 nucleotides.

13. The method of claim 2, characterized in that the gene or gene segment specific for sepsis and/or sepsis-like conditions is selected from the group consisting of SEQUENCE ID No. I.1 to SEQUENCE ID No. I.6242, as well as gene fragments thereof with 5-2000 or more, preferably 20-200, more preferably 20-80 nucleotides.

14. The method of claim 3, characterized in that the gene or gene segment specific for severe sepsis is selected from the group consisting of SEQUENCE ID No. II.1 to SEQUENCE ID No. II.130, as well as gene fragments thereof with 5-2000 or more, preferably 20-200, more preferably 20-80 nucleotides.

15. The method of claim 1, characterized in that at least 2 to 100 different cDNAs are used.

16. The method of claim 1, characterized in that at least 200 different cDNAs are used.

17. The method of claim 1, characterized in that at least 200 to 500 different cDNAs are used.

18. The method of claim 1, characterized in that at least 500 to 1000 different cDNAs are used.

19. The method of claim 1, characterized in that at least 1000 to 2000 different cDNAs are used.

20. The method of claim 1, characterized in that the cDNA of SEQUENCE ID No. III.1 to SEQUENCE ID No. III.4168, SEQUENCE ID No. I.1 to SEQUENCE ID No. I.6242 and SEQUENCE ID No. II.1 to SEQUENCE ID No. II.130 replaced by synthetic analoga as well as peptidonucleic acids.

21. The method of claim 20, characterized in that the synthetic analoga of the listed genes comprise 5-100, in particular approximately 70, base pairs.

22. The method one of claim 1, characterized in that a radioactive label, in particular ³²P, ¹⁴C, ¹²⁵I, ¹⁵⁵Eu, ³³P or ³H is used as detectable label.

23. The method of claim 1, characterized in that a non-radioactive label is used as detectable label, in particular a color- or fluorescence label, an enzyme label or immune label, and/or quantum dots or an electrically measurable signal, in particular the change in potential, and/or conductivity and/or capacity by hybridizations.

24. The method of claim 1, characterized in that the sample RNA and control RNA bear the same label.

25. The method of claim 1, characterized in that the sample RNA and control RNA bear different labels.

26. The method of claim 1, characterized in that the immobilized probes bear a label.

27. The method of claim 1, characterized in that the cDNA probes are immobilized on glass or plastics.

28. The method of claim 1, characterized in that the individual cDNA molecules are immobilized on the carrier material by means of a covalent binding.

29. The method of claim 1, characterized in that the individual cDNA molecules are immobilized onto the carrier material by means of adsorption, in particular by means of electrostatic and/or dipole-dipole and/or hydrophobic interactions and/or hydrogen bridges.

30. A method for in vitro detection of SIRS, comprising:
isolating sample peptides from a sample of a mammal;
labelling of the sample peptides with a detectable label;
contacting the labelled sample peptides with at least one antibody or its binding fragment, whereby the antibody binds a peptide or peptide fragment specific for SIRS;
contacting the labelled control peptides originating from healthy subjects, with at least one antibody or its binding fragment immobilized on a carrier in form of a microarray, whereby the antibody binds a peptide or peptide fragment specific for SIRS;
quantitative detection of the label signals of the sample peptides and the control peptides;
comparing the quantitative data of the label signals in order determine whether the genes or gene fragments specific for SIRS are more expressed in the sample than in the control, or less.

31. A method for in vitro detection of sepsis and/or sepsis-like conditions, comprising:
isolating sample peptides from a sample of a mammal;
labelling of the sample peptides with a detectable label;
contacting the labelled sample peptides with at least one antibody or its binding fragment, whereby the antibody binds a peptide or peptide fragment specific for sepsis and/or sepsis-like conditions;
contacting the labelled control peptides stemming from healthy subjects, with at least one antibody or its binding fragment immobilized on a carrier in form of a microarray, whereby the antibody binds a peptide or peptide fragment specific for sepsis and/or sepsis-like conditions;
quantitative detection of the label signals of the sample peptides and the control peptides; and
comparing the quantitative data of the label signals in order to be able to determine whether the genes or gene fragments specific for sepsis and/or sepsis-like conditions are more expressed in the sample than in the control, or less.

32. A method for in vitro detection of severe sepsis, comprising:
isolating sample peptides from a sample of a mammal;
labelling of the sample peptides with a detectable label;
contacting the labelled sample peptides with at least one antibody or its binding fragment, whereby the antibody binds a peptide or peptide fragment specific for severe sepsis;
contacting the labelled control peptides originating from healthy subjects, with at least one antibody or its binding fragment immobilized on a carrier in form of a microarray, whereby the antibody binds a peptide or peptide fragment specific for severe sepsis;
quantitative detection of the label signals of the sample peptides and the control peptides; and
comparing the quantitative data of the label signals in order to determine whether the genes or gene fragments specific for severe sepsis are more expressed in the sample than in the control, or less.

33. The method of claim 30, characterized in that the antibody is immobilized on an array in form of a microarray.

34. The method of claim 30, characterized in that it is formed as immunoassay.

35. The method of claim 30, characterized in that the method is used for early detection by means of differential diagnostics, for control of the clinic and therapeutic progress, for risk evaluation for patients as well as for post mortem diagnosis of SIRS and/or sepsis and/or severe sepsis and/or systemic infections and/or septic conditions and/or infections.

36. The method of claim 30, characterized in that the sample is selected from the following group: body fluids, in particular blood, liquor, urine, ascitic fluid, seminal fluid, saliva, puncture fluid, cell content, or a mixture thereof.

37. The method of claim 30, characterized in that cell samples are subjected a lytic treatment, if necessary, in order to free their cell contents.

38. The method of claim 30, characterized in that the mammal is a human.

39. The method of claim 30, characterized in that the peptide specific for SIRS is an expression product of a gene or gene fragment selected from the group consisting of SEQUENCE ID No. III.1 to SEQUENCE ID No. III.4168, as well as gene fragments thereof with 5-2000 nucleotides or more, preferably 20-200, more preferable 20-80 nucleotides.

40. The method of claim 31, characterized in that the peptide specific for sepsis and/or sepsis-like conditions is an expression product of a gene or gene fragment selected from the group consisting of SEQUENCE ID No. I.1 to SEQUENCE ID No. I.6242, as well as gene fragments thereof with 5-2000 nucleotides or more, preferably 20-200, more preferable 20-80 nucleotides.

41. The method according to one of claim 32, characterized in that the peptide specific for severe sepsis is an expression product of a gene or gene fragment selected from the group consisting of SEQUENCE ID No. II.1 to SEQUENCE ID No. II.130, as well as gene fragments thereof with 5-2000 or more, preferably 20-200, more preferably 20-80 nucleotides.

42. The method of claim 30, characterized in that at least 2 to 100 different peptides are used.

43. The method of claim 30, characterized in that at least 200 different peptides are used.

44. The method of claim 30, characterized in that at least 200 to 500 different peptides are used.

45. The method of claim 30, characterized in that at least 500 to 1000 different peptides are used.

46. The method of claim 30, characterized in that at least 1000 to 2000 different peptides are used.

47. The method of claim 30, characterized in that a radioactive label, in particular ³²P, ¹⁴C, ¹²⁵I, ¹⁵⁵Eu, ³³P or ³H is used as detectable label.

48. The method of claim 30, characterized in that a non-radioactive label is used as detectable label, in particular a color- or fluorescence label, an enzyme label or immune label, and/or quantum dots or an electrically measurable signal, in particular the change in potential, and/or conductivity and/or capacity by hybridizations.

49. The method of claim 30, characterized in that the sample peptides and control peptides bear the same label.

50. The method of claim 30, characterized in that the sample peptides and control peptides bear different labels.

51. The method of claim 30, characterized in that the probes used are peptides to which labelled antibodies are bound, which cause a change of signal of the labelled antibodies by change of conformation when binding to the sample peptides.

52. The method of claim 30, characterized in that the peptide probes are immobilized on glass or plastics.

53. The method of claim 30, characterized in that the individual peptide molecules are immobilized onto the carrier material by means of a covalent binding.

54. The method of claim 30, characterized in that the individual peptide molecules are immobilized on the carrier material by means of adsorption, in particular by means of electrostatic and/or dipole-dipole and/or hydrophobic interactions and/or hydrogen bridges.

55. The method of claim 30, characterized in that the individual peptide molecules are detected by means of monoclonal antibodies or their binding fragments.

56. The method of claim 30, characterized in that the determination of individual peptides by means of immunoassay or precipitation assay is carried out using monoclonal antibodies.

57. (canceled)

58. (canceled)

59. (canceled)
